(19) United States
(12) Patent Application Publication
(10) Pub. No.: US 2012/0015032 A1
(43) Pub. Date: Jan. 19, 2012

(54) COMBINATION PREPARATION COMPRISING INHIBITOR OF HMG-COA REDUCTASE AND ASPIRIN AND METHOD FOR MANUFACTURING THE SAME

(75) Inventors: Sung Wuk Kim, Gyeonggi-do (KR); Sung Soo Jun, Gyeonggi-do (KR); Young Gwan Jo, Daejeon (KR); Ja Seong Koo, Daejeon (KR); Sang Ouk Sun, Gwangju (KR)

(73) Assignee: HANALL PHARMACEUTICAL COMPANY, LTD., Daejeon (KR)

(21) Appl. No.: 12/672,630

(22) PCT Filed: Aug. 8, 2008
(86) PCT No.: PCT/KR08/04623

§ 371 Date: Feb. 8, 2010

(30) Foreign Application Priority Data
Aug. 13, 2007 (KR) 10-2007-0081206

(51) Int. Cl. A61K 9/28; A61P 9/00; A61P 9/10; A61K 31/60

(52) U.S. Cl. 424/474; 514/161; 514/163; 424/464

(57) ABSTRACT

The present invention relates to a chronotherapeutically combined pharmaceutical formulation for preventing and treating cardiovascular diseases, which is based on the principle of administering a plurality of drugs at certain time intervals (chronotherapy). Specifically, the combined pharmaceutical formulation comprises a HMG-CoA reductase inhibitor, such as simvastatin, and aspirin. Because the combined pharmaceutical formulation was developed based on the principle of administering drugs at certain time intervals, so-called chronotherapy, it shows an excellent effect of preventing or treating cardiovascular disease compared to those of the individual administration and simultaneous administration of the single preparations. Also, it is a once-daily dosage form which increases the medication compliance of patients. Particularly, even though the content of aspirin in the combined pharmaceutical formulation is reduced, the platelet aggregation inhibitory effect of aspirin in the combined pharmaceutical formulation is equal to that of the amount of aspirin used in the prior art, while the aspirin in the combined pharmaceutical formulation shows a antihypertensive effect. In addition, the chronotherapeutically combined pharmaceutical formulation allows the two drugs, which interact with each other, to be stored for a long period of time, and the combined pharmaceutical formulation ensures the human body-safety and efficacy of the two drugs.

### TECHNICAL FIELD

The present invention relates to a combined pharmaceutical formulation for preventing and treating cardiovascular diseases, which is based on the principle of administered at certain time intervals to have the optimal pharmacological activities, so-called chronotherapy, and more particularly to a combined pharmaceutical formulation, comprising aspirin and HMG-CoA reductase inhibitor.

### BACKGROUND ART

The term “cardiovascular disease (CVD)” refers to the group of diseases caused by dysfunctional conditions of the heart and blood vessels. As aging progresses, cardiac muscles weaken, and cholesterol, calcareous matter and the like is accumulated in coronary arteries to narrow arterial blood vessels, thus making smooth blood circulation difficult. The resulting diseases, including hyperlipidemia, stroke, myocardial infarction, arteriosclerosis, angina pectoris and the like, are referred to as cardiovascular diseases.

Hyperlipidemia refers to a condition in which plasma lipids such as cholesterol, triglyceride and the like are abnormally increased due to genetic factors, excessive intake of animal fats and carbohydrates, obesity or diabetes, renal disease, hypothyroidism, etc. Particularly, hyperlipidemia causes arterial thrombosis, leading to arteriosclerosis in which lipids are thickly accumulated along the vascular wall. Also, it reduces blood flow, thus causing ischemic heart disease, angina and myocardial infarction. Accordingly, the treatment of hyperlipidemia can prevent other cardiovascular diseases including arteriosclerosis and the like.

Although arteriosclerosis, stroke, angina, etc. are generally caused by hyperlipidemia, they are also frequently caused by another factor, that is, thrombus. The term “thrombus” refers to a blood clot, which is formed within a blood vessel due to the blood coagulation by the interaction between platelets and blood coagulation factors in the wound site of the blood vessel. This thrombus can be generated in any site of the body, and if it is generated in cerebral blood vessels or enters cerebral blood vessels to block the cerebral blood vessels, it can also cause fatal stroke.

In summary, cardiovascular diseases can be prevented and treated by lowering blood cholesterol levels and preventing thrombus formation.

A statin, lipid-lowering agent, HMG-CoA reductase inhibitor are a cholesterol synthesis inhibitor and is known as the most effective drug among hyperlipidemia therapeutic agents [Lancet 1995; 346: 750-753, Am J Cardiol 1998; 82: 57T-59T, AM J Caridol 1995; 76: 107C-112C, Hypertens Res 2003; 26: 699-704]. The statin, lipid-lowering agent, lower cholesterol levels required for the synthesis of bile acid and the like by inhibiting the activity of HMG-CoA reductase, which is involved in a process in which HMG-CoA is converted to mevalonic acid (that is, the rate-limiting step of cholesterol biosynthesis in liver cells). To compensate for this, the number of low density lipoprotein (LDL) receptors causing arteriosclerosis is increased and resulted in lowered concentration of the LDL levels through bringing the more LDL from blood. [Zocor tablet label—MSD®]. Particularly, because the synthesis of lipids in the liver becomes active after dinner in the early evening, The statin, lipid-lowering agents, have been administered in the early evening [Arterioscler Thromb 11: 816-826].

Also, the statin, lipid-lowering agents, show antihypertensive action of expanding blood vessels by increasing abnormal levels of endothelial nitric oxide synthases (eNOs), which are shown in hypertension patient, to normal levels [Am J Physiol Vol 281 Issue 5: F802-F809, 2001]. One of the most effective drugs for preventing thrombus formation is aspirin.

Aspirin has been widely used as an antipyretic and analgesic agent, and recently, has been used as a platelet aggregation inhibitor. When a low dose of aspirin is administered, acetylsalicylic acid irreversibly acetylates the cyclo-oxygenase (COX) to block the synthesis of thromboxane A2 (TXA-2), a platelet aggregation inducer, thus reducing platelets. Accordingly, aspirin prevents platelets in blood from adhering to each other, thus inhibiting platelet aggregation. In addition, antiplatelet effect and antihypertensive effect of aspirin vary depending on the administration time. The human body keeps a constant rhythm. In the daytime, cyclooxygenase-2 (COX-2 enzyme) associated with inflammation is more than cyclooxygenase-1 (COX-1 enzyme) which is mainly involved in the synthesis of thromboxane-A2. Namely, when aspirin is administered in the morning, it is difficult to strongly block the platelet aggregation inducer TXA-2. On the contrary, in the nighttime, the COX-1 enzyme is more than the COX-2 enzyme. If the concentration of aspirin in blood is high in the nighttime, aspirin shows the highest inhibitory activity on platelet aggregation by irreversible binding to the COX-1 enzyme, until platelets are killed or until the next day when fresh platelets are actively produced. Also, there is a report that, if aspirin is administered in the morning, it has no antihypertensive effect, but if it is administered before bedtime, it shows the effects of antihypertensive effect and preventing hypertension. [Administration Time-Dependent Effect of Aspirin on Blood Pressure in Untreated Hypertensive Patients, Hypertension 2003; 41; 1259-1267] [Aspirin at bedtime best time to cut blood pressure May 15, 2002, The Annual Scientific Meeting of the American Society of Hypertension; Dr. Ramon C. Hermida of the university of Vigo, Spain] [Differing Administration time-dependent effects of low-dose aspirin on ambulatory blood pressure in dipper and non-dipper hypertensive patients, Ramon C Hermida et al, P-151.][Aspirin at Bedtime Lowers Blood Pressure, May 14, 2008, The Annual Scientific Meeting of the American Society of Hypertension; Dr. Ramon C. Hermida of the university of Vigo].

Aspirin can sufficiently prevent thrombus formation in an amount of merely 20-40 mg. Although, the amount of aspirin administered orally is more than 40 mg, the antiplatelet effect of aspirin doesn't increase. The remaining amount of aspirin shows anti-inflammatory and analgesic activities by inhibiting the COX-2 enzyme activity. Accordingly, aspirin has a thrombus formation-preventing effect only in a small amount [Drug Insight: Aspirin Resistance-Fact or Fashion, Carlo Patrono et al., Nat Clin pract Cardiovasc Med. 2007; 4(1):42-50][Use of Low-Dose Aspirin in TIA and Thrombotic Stroke; Ask the Experts about Cardiovascular from Medscape Internal Medicine; Gerald W. Smetana, MD Division of General Medicine and Primary Care, Beth Israel Deaconess Medical Center, Harvard Medical School]. Statin, lipid-lowering agents, include simvastatin, atorvastatin, pravastatin, fluvastatin, rosuvastatin, cerivastatin, and salts or isomers thereof. Among them, simvastatin and atorvastatin are most widely used in the world.

The prior art relating to the combined pharmaceutical formulation for preventing and treating cardiovascular disease, proposed in the present invention, is as follows.

U.S. Pat. No. 6,235,311 discloses a pharmaceutical composition, comprising a statin drug and aspirin, and a preparation method thereof. In said patent, a composition for treating cardiovascular disease is provided in the form of preparations such as a bilayer tablet, a press-coated tablet and the like in order to prevent the interaction between aspirin and a statin drug. However, the composition disclosed in said patent is predictable from natural therapeutic effects of two drugs and it differs from the composition preparation of the present invention, which is designed so as to be suitable for administration at a specific time. In addition, unlike the present invention, said patent does not contain specific test examples, such as stability tests, dissolution tests, animal tests and the like, and thus it is difficult to predict the exact therapeutic effect of the composition.

U.S. Pat. No. 6,576,256 discloses a method of treating a patient at a cardiovascular risk, comprising administering a composition of single preparation type, comprising a HMG-CoA reductase inhibitor, an angiotensin converting enzyme inhibitor, aspirin, vitamin B6, vitamin B12 and folic acid as a cholesterol-lowering agent, once a day. However, said patent does not contain specific formulations and test examples.

Korean Patent No. 10-0646576 discloses a combination pellet for preventing arteriosclerosis in hyperlipidemia patients, comprising a HMG-CoA reductase inhibitor and enteric-coated aspirin. However, said patent has problems in terms of preparation time and stability, because the main components must be coated by dissolving or suspending them, and furthermore, may have problems in terms of content uniformity and yield due to the coating process.

### DISCLOSURE OF INVENTION

### Technical Problem

It is an object of the present invention to provide a combined pharmaceutical formulation for preventing and treating cardiovascular disease, comprising a HMG-CoA reductase inhibitor and aspirin. More specifically, the object of the present invention is to ensure excellent clinical therapeutic effects compared to those of the individual or simultaneous administration of single preparations, by adjusting drug release pattern such that each of the drugs is released at the time at which each drug shows the highest effect, based on the theory of chronotherapy. Another object of the present invention is to provide a combined pharmaceutical formulation, in which drugs show the highest effect through pharmaceutical technology, even though the currently used dosages of the drugs are reduced, and thus the combined pharmaceutical formulation has drug effects equal to those of the currently used dosages and can reduce side effects. Still another object of the present invention is to develop said combined pharmaceutical formulation into a once-daily dosage form in order to increase the medication compliance of patients. Yet another object of the present invention is to provide a dosage form, a formulation and method of manufacturing, which can maintain drugs stable for a long period of time.

### Technical Solution

The present invention relates to a combination drug delivery system comprising, as active ingredients, a HMG-CoA reductase inhibitor and aspirin, wherein the drug delivery system is designed so as to prevent the state in which the HMG-CoA reductase inhibitor and aspirin are simultaneously released in vivo after administration.

The drug delivery system is designed in consideration of gastrointestinal transit time of drugs, the solubility of each drug, intestinal permeability, effects on diet and the like.

The drug delivery system consists of a section, comprising the HMG-CoA reductase inhibitor as an active ingredient, and a section comprising aspirin.

The HMG-CoA reductase inhibitor is characterized in being selected from the group consisting of simvastatin, atorvastatin, pravastatin, fluvastatin, rosuvastatin, cerivastatin, pharmaceutically acceptable salts thereof and isomers thereof.

### Advantageous Effects

A chronotherapeutically combined pharmaceutical formulation according to the present invention is effective in preventing and treating hyperlipidemia and arteriosclerosis, which are fatal risk factors in cardiovascular disease patients, because it perfectly completes the pharmacological and clinical therapeutic effects of a HMG-CoA reductase inhibitor (e.g., simvastatin) and aspirin, which decrease when single preparations of the drugs are simultaneously administered. Also, the present invention relates to a combined pharmaceutical formulation of a HMG-CoA reductase inhibitor and aspirin, which is administered once a day in the evening, and thus contributes to improve the medication compliance of increasing old-age patients more than expectation.

Furthermore, the combined pharmaceutical formulation of the present invention is the first combined pharmaceutical formulation comprising simvastatin and aspirin, which was found to show an antihypertensive effect when it was administered in the evening.

In addition, the combination drug delivery system of the present invention is considered to be an optimal drug delivery system for the development of combined pharmaceutical formulations.

Finally, because the present invention relates to a combined pharmaceutical formulation of components having different pharmacological activities, it can offset side effects and reduce the risk factors of development of cardiovascular disease. Thus, the present invention is very efficient in economic terms, because it can reduce long-term prevention cost and packaging cost, and reduce the time for highly educated manpower to prescribe and prepare medicines.

### DESCRIPTION OF DRAWINGS

FIG. 1 shows the result of dissolution test of aspirin in combined pharmaceutical formulations of Examples 1, 2 and 4.

FIG. 2 shows the result of dissolution test of simvastatin in combined pharmaceutical formulations of Examples 1, 2 and 4.

FIG. 3 shows the result of dissolution test of simvastatin and aspirin in combined pharmaceutical formulations of Examples 7 and 9, measured in test examples.

FIG. 4 shows the result of dissolution test of aspirin in combined pharmaceutical formulations of Examples 10, 11, 12 and 13, measured in test examples.

FIG. 5 shows the result of dissolution test of simvastatin and aspirin in combined pharmaceutical formulations of Examples 17 and 21, measured in test examples.

FIG. 6 shows the result of dissolution test of atorvastatin, fluvastatin and aspirin in combined pharmaceutical formulations of Examples 25 and 26, measured in test examples.

FIG. 7 shows the result of dissolution test of simvastatin and aspirin in combined pharmaceutical formulations of Examples 33 and 34, measured in test examples.

### BEST MODE

The present invention, in a chronotherapeutic combined drug delivery system comprising, as active ingredients, a HMG-CoA reductase inhibitor and aspirin, is characterized in that the active ingredients show different dissolution patterns and absorption patterns in vivo. The drug delivery system of the present invention can be designed in a manner such that aspirin is dissolved and absorbed in the stomach and the IIMG-CoA reductase inhibitor is dissolved and absorbed in the oral mucosa or is dissolved and absorbed faster than aspirin in stomach, or is absorbed later than aspirin in the stomach, the small intestines or the large intestines. The reverse order is also possible.

Specifically, after the HMG-CoA reductase inhibitor is released in advance, the aspirin component is preferably released within 15 minutes to 4 hours after the release of HMG CoA reductase inhibitor. Also, as measured in a dissolution test in simulated intestinal fluid containing 1% sodium lauryl sulfate, it is preferable that at 30 minutes after the start of dissolution, not less than 75% of the HMG-CoA reductase inhibitor component is dissolved and less than 40% of the aspirin component is dissolved.

On the contrary, after the aspirin component is released in advance the IIMG-CoA reductase inhibitor component is preferably released within 15 minutes to 4 hours after the release of aspirin Also, as measured in a dissolution test in simulated intestinal fluid containing 1% sodium lauryl sulfate, it is preferable that at 15 minutes after the start of dissolution, not less than 70% of the aspirin component is dissolved and less than 40% of the HMG-CoA reductase component is dissolved.

The principle of administering drugs at certain time intervals (so called Chronotherapy) in the present invention can be explained as follows:

1. The HMG-CoA reductase inhibitor as an active ingredient is administered in an amount of 0.5-80 mg once a day in the evening. The reason is that HMG-CoA reductase inhibitors including simvastatin are recommended to be administered in the evening, because the synthesis of lipids in the liver is active after dinner in the early evening [Arterioscler Thromb 11: 816-826].

2. Aspirin, which is administered for the prevention of thrombus formation, is commonly administered in an amount of 75-300 mg once a day, and it is marketed in the form of enteric-coated tablets, sustained-release tablets or buffered tablets in order to prevent gastrointestinal bleeding caused by the long-term administration thereof.

3. The effects of aspirin are distinctly different between administration in the morning and in the evening due to biochemical rhythms such as the major formation time of in vivo enzymes. Namely, when aspirin is administered in the morning, it has a reduced effect on the prevention of thrombus formation and has no antihypertensive effect.

4. When an enteric-coated tablet of aspirin, which is currently used, is administered simultaneously with a HMG-CoA reductase inhibitor, it is difficult to show the perfect effects of the two components, because the two components are unstable due to the interaction therebetween.

5. It can be seen that, when the combined pharmaceutical formulation of the present invention is administered, it shows the optimal effects of lowering blood lipid level and inhibiting platelet aggregation, because the dissolution and absorption times of the HMG-CoA reductase inhibitor and aspirin are different.

Another major object to be achieved in the present invention is to reduce the amount of aspirin commonly used. The thromboxane-A2 production inhibitory activity of aspirin does not increase in proportion to the amount of aspirin. Namely, aspirin can show sufficient effects, even when it is administered only in an amount of 20-40 mg. However, due to the genetic difference in the amount of the COX-1 enzyme and the use habits of patients, aspirin is currently mainly prescribed in an amount of 75-300 mg. According to the present invention, aspirin can exhibit an excellent platelet aggregation inhibitory effect only in an amount of 20-40 mg, because it is formulated to bind to the produced COX-1 enzyme through the principle of administering drugs at different times, such that it irreversibly binds to the produced COX-1 enzyme.

Still another object to be achieved in the present invention is to prepare a once-daily dosage form based on the chronotherapy in order to increase the medication compliance of patients. Thus, the time and economic expenditures of manufacturers can be reduced and the medication expenditure of patients can be reduced.

Yet another object to be achieved in the present invention is to develop a preparation for oral administration in order to inhibit the degradation caused by interaction between a HMG-CoA reductase inhibitor (such as simvastatin) and aspirin.

The combined pharmaceutical formulation according to the present invention is characterized in that, because it is based on the chronotherapy, the blood lipid concentration-lowering effect and platelet aggregation inhibitory effect thereof are significantly excellent compared to those of the individual administration and simultaneous administration of prior single preparations. Particularly, even though the amount of aspirin used is reduced, the platelet aggregation inhibitory effect of aspirin in the combined pharmaceutical formulation of the present invention is equal to that of the amount of aspirin used in the prior art, and the side effects of aspirin in the combined pharmaceutical formulation are reduced.

Furthermore, the present invention provides a chronotherapeutic combined drug delivery system designed such that drugs, which are unstable due to the interaction with each other, such as a HMG-CoA reductase inhibitor and aspirin, are not degraded in vivo due to the interaction therebetween.

In addition, according to the present invention, the pharmaceutical stability of a IIMG-CoA reductase inhibitor and aspirin are improved such that they can be stored for a long period of time, and the human body-safety and efficacy of the two drugs are ensured.

Finally, the combined pharmaceutical formulation according to the present invention is administered once a day in order to increase the medication compliance of patients and reduce the time and economic expenditures of manufacturers and the medication expenditure of patients.

Hereinafter, the present invention will be described in further detail, but the scope of the present invention is not limited to the description.

The present invention allows a HMG-CoA reductase inhibitor and aspirin to be dissolved and absorbed at different sites after administration. The combination drug delivery system according to the present invention is a system designed such that, even though the two components are absorbed at the same site, they can be released at different times.

Examples of the HMG-CoA reductase inhibitor include simvastatin, atorvastatin, pravastatin, fluvastatin, rosuvastatin, cerivastatin, pharmaceutically acceptable salts thereof and isomers thereof.

In order for the drugs to have different dissolution patterns and absorption characteristics, each of the drugs may have release mechanisms, such as fast disintegrating properties, fast release properties, effervescent properties, mucoadhesive properties, gastric-retentive properties, delayed-release properties, sustained-release properties, colon delivery properties, etc.

Also, the combined pharmaceutical formulation of the present invention can be embodied in all orally administrable dosage form within the range in which the stability thereof is ensured. Examples of the preparations include tablets, such as uncoated tablets, film-coated tablets, enteric-coated tablets, sustained-release tablets, multilayer tablets, press-coated tablets, and Oros tablets, granules, pellets, capsule formulations prepared by filling mini-tablets, powders or pills in capsules, powders, pills, etc., as well as kits allowing drugs to be simultaneously administered.

The present invention can be formulated such that aspirin is dissolved and absorbed faster than the HMG-CoA reductase inhibitor. Alternatively, it can be formulated such that the HMG-CoA reductase inhibitor is dissolved and absorbed faster than aspirin.

The drug component, which is dissolved and absorbed faster than the other component, can be prepared by using a conventionally used diluent (filler), binder, disintegrant or lubricant, and if necessary, pharmaceutical additives having rapid disintegrating properties may be used.

The drug component, which is dissolved and absorbed delayed than the other component, can be prepared by a conventional mixing, wet granulation, dry granulation or coating process using at least one release controlling material selected from the group consisting of water soluble polymers, water insoluble polymers, enteric polymers, oils and gums. The resulting particles or granules can be tableted individually or in combination with the particles or granules containing the component, which is released faster, or the particles or granules can be filled in capsules.

Also, the powders or granules, which are released faster and slower, respectively, may be used to embody preparations, such as multilayer tablets, press-coated tablets, etc.

However, the inventive preparations based on the principle of administering drugs at certain time intervals (chronotherapy) are not limited to the above-described preparations.

The present invention relates to a technology of manufacturing a combined pharmaceutical formulation using pharmaceutically acceptable additives, for example, diluents, binders, disintegrants, lubricants, stabilizers and film coating agents, such that the component, which is dissolved and absorbed in advance, is rapidly disintegrated and released and absorbed in the gastrointestinal tract.

The present invention relates to a technology of manufacturing a combined pharmaceutical formulation using at least one release controlling material selected from the group consisting of water-soluble polymers, water-insoluble polymers, enteric polymers, oils and gums, together with pharmaceutically acceptable diluents, binders, disintegrants, lubricants and stabilizers, such that the component, which is dissolved and absorbed later, is dissolved and absorbed, after the release thereof is intentionally delayed during a given time.

In the present invention, the component, which is dissolved and absorbed later, is formulated using a release-controlling substance in an amount of 0.1-100 parts by weight, preferably 0.1-20 parts by weight, and more preferably 0.2-10 parts by weight, based on 1 parts by weight of the drug.

If the release-controlling substance is used in an amount of less than 0.1 parts by weight, the intended delay time cannot be achieved, and if it exceeds 100 parts by weight, the delay time becomes excessively long, thus making it difficult to expect the highest effect of each of the drugs.

As the water-soluble polymer, water-soluble cellulose ether selected from the group consisting of methylcellulose, hydroxypropylcellulose and hydroxypropylmethylcellulose, a water-soluble polyvinyl derivative selected from the group consisting of polyvinyl pyrrolidone and polyvinyl alcohol, or an alkylene oxide polymer selected from the group consisting of polyethylene glycol and polypropylene glycol, may be used.

Also, as the water-insoluble polymer, a water-insoluble cellulose ether selected from the group consisting of ethyl cellulose and cellulose acetate, or a water-insoluble acrylic acid copolymer selected from the group consisting of an ethylacrylate/methylmethacrylate/trimethylammonium chloride ethyl methacrylate copolymer (e.g., Eudragit RS or RL, Degussa) and a methylmethacrylate/ethylacrylate/trimethylammonium chloride ethyl copolymer (e.g., Eudragit NE30D, Degussa), may be used.

Also, the enteric polymer may be one or two or more selected from the group consisting of: an enteric cellulose derivative selected from hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose benzoate phthalate, cellulose propionate phthalate, methylcellulose phthalate, carboxymethylethylcellulose and ethylhydroxyethylcellulose phthalate; an enteric acrylic acid copolymer selected from a styrene/acrylic acid copolymer, a methylacrylate/acrylic acid copolymer, a methylacrylate/methacrylic acid copolymer, a butylacrylate/styrene/acrylic acid copolymer, a methacrylic acid/ethylmethacrylate copolymer (e.g., Eudragit L 100 or Eudragit S, Degussa), a methacrylic acid/ethylayerylate copolymer (e.g., Eudragit L 100-55, Degussa) and a methylacrylate/methacrylic acid/octylacrylate copolymer; an enteric maleic acid copolymer selected from a vinylacetate/maleic anhydride copolymer, a styrene/maleic anhydride copolymer, a styrene/maleic monoester copolymer, a vinylmethylether/maleic anhydride copolymer, an ethylene/maleic anhydride copolymer, a vinylbutylether/maleic anhydride copolymer, an acrylonitrile/methylacrylate/maleic anhydride copolymer and a butylacrylate/styrene/maleic anhydride copolymer; and an enteric polyvinyl derivative selected from polyvinylalcohol phthalate, polyvinylacetal phthalate, polyvinylbutyrate phthalate and polyvinylacetacetal phthalate.

Also, the oil substance may be selected from the group consisting of fatty acids and fatty acid esters, fatty acid alcohols, and waxes and the like. Specifically, examples of the fatty acid and fatty acid esters include glyceryl palmitostearate, glyceryl stearate, glyceryl behenate, cetyl palmitate, glyceryl monooleate, and stearic acid, examples of the fatty acid alcohols include cetostearyl alcohol, cetylalcohol and stearylalcohol, and examples of the waxes include carnauba wax, beeswax and microcrystalline wax.

Also, the gum may be selected from the group consisting of guar gum, locust bean gum, tragacantha, carrageenan, acacia gum, arabia gum, gellan gum, xanthan gum, pectin and the like.

In the present invention, it can be formulated by using, in addition to the above-described substances and active ingredients, pharmaceutically acceptable diluents including starch, microcrystalline cellulose, lactose, glucose, mannitol, alginate, alkaline earth metal salt, clay, polyethylene glycol and dicalcium phosphate, within the range in which the effects of the present invention are not impaired.

Also, as the binder that is used in the combined pharmaceutical formulation of the present invention, starch, microcrystalline cellulose, mannitol, lactose, polyethylene glycol, polyvinyl pyrrolidone, hydroxypropylmethyl cellulose, hydroxypropyl cellulose, natural gum, synthetic gum, copovidone, gelatin and the like may be used.

Also, as the disintegrant that is used in the combined pharmaceutical formulation of the present invention, starch or modified starch, such as sodium starch glycolate, corn starch, potato starch and pre-gelatinized starch; clay such as bentonite, montmorillonite or veegum; cellulose such as microcrystalline cellulose, hydroxypropylcellulose and carboxymethylcellulose; alginate such as sodium alginate and alginic acid; crosslinked cellulose such as croscarmellose sodium; gum such as guar gum and xanthan gum; a crosslinked polymer such as crospovidone; and effervescent materials such as sodium bicarbonate and citric acid may be used in a combination.

Also, as the lubricant that is used in the combined pharmaceutical formulation of the present invention, talc, stearic acid or salts thereof, sodium lauryl sulfate, hydrogenated vegetable oil, sodium benzoate, sodium stearyl fumarate, glyceryl monostearate, polyethylene glycol and the like may be used. In addition, other various pharmaceutically acceptable additives such as colorants and perfumes may be used in the combined pharmaceutical formulation of the present invention.

The range of additives in the present invention is not limited to the above-described additives, and the above-described additives are contained in the combined pharmaceutical formulation in conventional amounts.

If necessary, a film-shaped coating layer may be formed on the outer surface of the above-described tablet layer in the combined pharmaceutical formulation of the present invention. Herein, the film comprises a film-forming agent, a film-forming aid or a mixture thereof.

The coating agent may be selected from the group consisting of cellulose derivatives such as hydroxypropylmethyl cellulose and hydroxypropyl cellulose, sugar derivatives, polyvinyl derivatives, waxes, fats and gelatins, and the coating aid may be one or a mixture of at least two selected from the group consisting of polyethylene glycol, triethyl citrate, glyceride, titanium oxide, talc and diethyl phthalate.

The dose of the HMG-CoA reductase inhibitor in the combined pharmaceutical formulation of the present invention is in the range of 0.1-100 mg/tablet, and the dose of aspirin is in the range of 10-1000 mg. Preferably, the dose of the HMG-CoA reductase inhibitors is in the range of 0.5-80 mg/tablet, and the dose of aspirin is in the range of 20-700 mg/tablet. Particularly, in, the present invention, the amount of aspirin used is less than 75 mg, and most preferably 20-40 mg.

Each step of the method for manufacturing the inventive combined pharmaceutical formulation comprising a HMG-CoA reductase inhibitor and aspirin will be described in detail.

The first step is a step of obtaining particles or granules through conventional processes for oral solid preparations, including mixing a drug, which is released in advance, with pharmaceutically acceptable conventional additives, and kneading, drying and granulating the mixture.

The second step is a step of obtaining particles or granules by mixing a drug, which is delayed released, with one or a mixture of at least two selected from the group consisting of water-soluble polymers, water-insoluble polymers, enteric polymers, oils and gums along with pharmaceutically acceptable conventional additives, and subjecting the mixture to a wet granulation, dry granulation or coating process. In this step, the particles or granules can be obtained either by performing the mixing process in combination with the coating process or by performing the first and second coating processes.

The third step is a step of obtaining a preparation for oral administration by mixing the particles or granules, obtained each of the first and second steps, with a pharmaceutically acceptable excipient, and compressing the mixture into tablets or filling the mixture in capsules.

If the drug component that is released in advance is aspirin, the drug component that is delayed released is a HMG-CoA reductase inhibitor, and if the component that is released in advance is a HMG-CoA reductase inhibitor, the component that is delayed released is aspirin.

The combined pharmaceutical formulation of the present invention is manufactured through the above-described method and can be finally formulated in the following manner.

(A) Uncoated Tablet and Film-Coated Tablet The particles or granules, obtained in the first and second steps, are compressed into a tablet by a certain weight, without any additional processing. Alternatively, the particles or granules are compressed into a tablet, after a release-controlling substance is added thereto (uncoated tablet). The obtained tablet may, if necessary, be coated with a film in order to improve the stability or properties of the tablet (film-coated tablet). Also, the film-coated tablet is manufactured by preparing the tablet containing the component that is released earlier, and then suspending or dissolving the component or mixtures that is released later in a film coating solution and coating the solution or suspension on the tablet.

(B) Preparation of Multilayer Tablet

The granules, obtained in the first step and second step or obtained by additional coating, can be compressed into a bilayer tablet using a multilayer compressor. If necessary, placebo granules containing no active ingredient may be prepared separately and inserted as a middle layer to prepare a multilayer tablet having three layers or more, and the multilayer tablet may be subjected to an additional coating process to prepare a coated multilayer tablet. The placebo granules can be prepared by performing a mixing or granulation process using pharmaceutically acceptable additives (e.g., an excipient, a binder, a disintegrant, a lubricant or a colorant) at suitable ratios.

(C) Preparation of Press-Coated Tablet

The granules obtained in the second step are compressed into an inner core without any additional processing, or the granules obtained by additional coating in the second step are compressed into an inner core, after they are coated with a release-controlling substance and dried. The inner core, together with the granules obtained in the first step, is compressed into a press-coated tablet by using a compressor and is subjected to a coating process to prepare a coated press-coated tablet. If necessary, additional coating on the inner core can be performed.

(D) Preparation of Capsule Formulation

The granules obtained in the first step are used without any additional processing. Alternatively, the granules are coated with a release-controlling substance and dried. Then, the granules obtained in the first step, together with the granules obtained in the second step, are placed in a capsule-filling machine, in which the granules are filled in a certain size capsule in amounts corresponding to the effective amounts of the drugs, thus preparing a capsule formulation. Alternatively, the granules obtained in each of the first and second steps may be formulated in the form of tablets having a general size, mini-tablets or pellets, and the prepared tablets or pellets may be filled in a capsule, thus preparing a capsule formulation.

(E) Preparation of Kit Formulation

The early-release drug-containing preparation obtained in the first step, together with the late-release drug-containing preparation obtained in the second step, may be placed in a foil, a blister, a bottle and the like, thus preparing a kit which allows the drug preparations to be administered simultaneously.

When the inventive combined pharmaceutical formulation as described above is administered once a day, particularly in the evening, a synergistic effect resulting from the release of drugs at different times can be obtained, and thus the combined pharmaceutical formulation can exhibit an excellent effect of preventing or treating cardiovascular disease. The dosage of the inventive combined pharmaceutical formulation into the human body can be suitably selected depending on the absorption, metabolism and excretion rate of active ingredients in vivo and the patient's age, sex, condition and the like.

### EXAMPLES

Hereinafter, the present invention will be described in detail with reference to examples, but the scope of the present invention is not limited to these examples.

### Example 1

### Film-Coated Tablet of Simvastatin-Aspirin

(1) Simvastastin Granules

According to the components and contents shown in Table 1 below, simvastatin, lactose and microcrystalline cellulose were weighed, sieved through sieve No. 20 and mixed with each other in a double cone mixer for 5 min to prepare a mixture. Meanwhile, hydroxypropylcellulose and citric acid were dissolved in purified water to prepare a binder solution. The mixtures were placed in a fluidized bed granulator, in which it was granulated by adding the binder solution thereto. In the granulation process, a high-shear mixer may also be used. The fluidized bed granulator was GPCG-1 (Glatt, Germany) equipped with a top-spray system. After the mixture was placed in the granulator, it was preheated in the following conditions: air floe: 80 m³/hr; inlet air temperature: 40° C.; and filter shaking (delta P filter<500 pa) carried out for 5 seconds/30 seconds in the asynchronous mode. When the temperature of the mixture reached 35° C. in the preheating process, the binding solution was sprayed onto the mixture at a rate of 1.0-10 g/min to granulate the mixture, and the pressure of atomizing air was adjusted in the range of 1.0-2.0 bar to control the spray angle of the binding solution. Because particles were produced with the progression of the process, the air flow was increased from 80 m³/hr to 100 m³/hr or 120 m³/hr, and filter shaking (delta P<4000 pa) was carried out for 5 seconds/min in the synchronous mode in order to prevent loss.

After completion of the granulation process, the granulated material was dried in a fluidized bed dryer.

The fluidized bed dryer used was GPCG-1 (Glatt, Germany), and after the granulated material was placed in the dryer, it was treated in the following conditions: Air flow: 120 m³/hr; inlet air temperature: 65° C.; and filter shaking (delta P filter<4000 pa) carried out for 5 seconds/30 seconds in the asynchronous mode. When the temperature of the granulated material reached 40° C., a sample was collected from the material. When the sample satisfied a loss of drying (LOD) of less than 2.5%, the drying process was completed, and when the LOD exceeded 2.5%, the drying process was further carried out, a sample was collected and measured for the LOD, and the drying process was completed if the sample satisfied the weight loss criteria.

After completion of the drying process, the dried material was sieved using an F-type oscillator equipped with a 1.0 mm sieve, and the sieved material, butylhydroxyanisol, Crospovidone and lactose were placed in a double cone mixer, in which they were mixed with each other for 10 minutes. Then, stearic acid was added to and mixed with the mixture for 4 minutes, thus preparing simvastatin granules.

(2) Aspirin Granules

According to the components and contents shown in Table 1 below, aspirin and talc were mixed with each other in a double cone mixer for 5 minutes to prepare a mixture. Meanwhile, hydroxypropylmethylcellulose (6 cps) and PEG 6,000 were dissolved in an ethanol-methylene chloride solution to prepare a first coating solution. The mixture was placed in a fluidized bed coating machine, in which it was coated with the first coating solution.

As the fluidized bed coating machine, GPCG-1 (Glatt, Germany) equipped with a bottom-spray system was used. In the coating machine, a plate to be controlled according to the size of granules was a B or C type, the partition gap was 15-25 cm, and a spray nozzle was 1 mm. After the mixture was placed in the machine, it was preheated in the following conditions: air flow: 100 m³/hr; inlet air temperature 45-60° C.; product temperature 35-50° C.; and filter shaking (delta P filter<500 pa) carried out for 5 seconds/30 seconds in the asynchronous mode. When the temperature of the mixture in the preheating process reached 35° C., the film coating solution was sprayed onto the mixture at a rate of 1-5 g/min to coat the mixture with the coating solution, and the pressure of atomizing air was adjusted in the range of 1.0-1.5 bar to control the spray angle of the coating solution. During the coating process, the temperature of the material was maintained at 34-40° C., and after completion of the coating process, the coated material was dried for about 1 hour and surface-treated, while the temperature of the coated material was maintained at 40° C.

After completion of the first coating process, the coated material was subjected to a second coating process using an enteric polymer, methacrylic acid/ethylacrylate copolymer (e.g., commercially available under the trade name of Eudragit L 100-55, Degussa, Opadry, Colorcon), in the same conditions as in the first coating process. Microcrystalline cellulose, pre-gelatinized starch, Copovidone and colloidal silicon dioxide were sieved through sieve No. 20 and mixed with the coated material for 10 minutes. After completion of the mixing, stearic acid sieved through sieve No. 35 was mixed with the mixture for 4 minutes, thus preparing aspirin granules.

(3) Tableting and Coating

The simvastatin granules of Example 1-(1) and the aspirin granules of Example 1-(2) were mixed with each other, and then compressed into tablets in a rotary compressor (MRC-30, Sejong Machinery Co., Korea). The resulting tablets were coated with a solution of hydroxypropylmethylcellulose 2910, hydroxypropylcellulose, titanium oxide, talc, iron oxide red and iron oxide yellow in ethanol and purified water in conventional tablet coating conditions.

### Example 2

### Film-Coated Tablet of Simvastatin-Aspirin

(1) Simvastatin Granules

According to the components and contents shown in Table 1 below, simvastatin, lactose and microcrystalline cellulose weighed, sieved through sieve No. 20 and mixed with each other in a double cone mixer for 5 minutes to prepare a mixture. Meanwhile, hydroxypropylcellulose and citric acid were dissolved in purified water to preparing a binding solution. The mixture was placed in a high-shearshear mixer, in which it was granulated by adding the binding solution thereto. The granules were dried in the same conditions as the fluidized bed drying conditions of Example 1, and then the dried granules were placed in a fluidized bed granule coating machine, in which the granules were coated with an enteric polymer, methacrylic acid/ethylacrylate copolymer (e.g., commercially available under the trade name of Eudragit L 100-55, Degussa, Opadry, Colorcon), in the same conditions as the aspirin granule coating conditions of Example 1. After completion of the coating process, microcrystalline cellulose, butylhydroxyanisol and stearic acid, sieved through sieve No. 35, were added to and mixed with the coated material for 4 minutes, thus preparing simvastatin granules.

(2) Aspirin Granules

According to the components and contents shown in Table 1 below, aspirin, microcrystalline cellulose, pre-gelatinized starch and colloidal silicon dioxide were mixed with each other in a mixer for 20 minutes. After completion of the mixing, stearic acid sieved through sieve No. 35 was added to and mixed with the mixture for 4 minutes, thus preparing aspirin granules.

(3) Tableting and Coating

The simvastatin granules of Example 2-(1) and the aspirin granules of Example 2-(2) were mixed with each other, and then compressed into tablets in a rotary compressor (MRC-3030, Sejong Machinery Co., Korea). The resulting tablets were coated with a solution of hydroxypropylmethylcellulose 2910, hydroxypropylcellulose, titanium oxide, talc, iron oxide red and iron oxide yellow in ethanol and purified water in conventional tablet coating conditions.

### Example 3

### Film-Coated Tablet of Atorvastatin-Aspirin

(1) Atorvastatin Granules

According to the components and contents shown in Table 1 below, atorvastatin calcium, microcrystalline cellulose and D-mannitol were sieved through sieve No. 20, and then mixed with each other in a double cone mixer for 20 minutes. After completion of the mixing process, the mixture was placed in a high-shear mixer, in which it was kneaded with a binding solution of hydroxypropylcellulose and citric acid in purified water for 4 minutes. After completion of the kneading process, the kneaded material was dried in a drying ovendrying-oven and then sieved. Then, sodium starch glycolate, butylhydroxyanisol and colloidal silicon dioxide were added to and mixed with the dried material for 10 minutes, and stearic acid sieved through sieve No. 35 was added to and mixed with the mixture for 4 minutes, thus preparing atorvastatin granules.

(2) Aspirin Granules

According to the components and contents shown in Table 1 below, aspirin granules were prepared in the same manner as in Example 1.

(3) Tableting and Coating

The atorvastatin granules of Example 3-(1) and the aspirin granules of Example 3-(2) were mixed with each other, and then compressed into tablets in a rotary compressor (MRC-30, Sejong Machinery Co., Korea). The resulting tablets were coated with a solution of hydroxypropylmethylcellulose 2910, hydroxypropylcellulose, titanium oxide, talc, iron oxide red and iron oxide yellow in ethanol and purified water in conventional tablet coating conditions.

### Example 4

### Film-Coated Tablet of Simvastatin-Aspirin

(1) Simvastatin Coating Solution According to the components and contents shown in Table 1 below, simvastatin, hydroxypropylmethylcellulose, talc, polyethyleneglycol 6,000 and butylhydroxyanisol were dissolved in an ethanol-methylene chloride solution to prepare a coating solution.

(2) Aspirin Tablet

According to the components and contents shown in Table 1 below, aspirin, microcrystalline cellulose, pre-gelatinized starch, alginic acid, hydroxypropylmethylcellulose and colloidal silicon dioxide were sieved through sieve No. 20, and then mixed with each other for 20 minutes. After completion of the mixing process, stearic acid sieved through sieve No. 35 was added to and mixed with the mixture for 4 minutes. The resulting mixture was compressed into tablets.

(3) Coating

The aspirin tablets of Example 4-(2) were placed in a high-coater (Sejong Machinery Co., Korea), in which the tablets were coated with the simvastatin coating solution of Example 4-(1). After completion of the coating process, the coated tablets were coated with a coating solution, obtained by dissolving hydroxypropylmethylcellulose 2910, polyethyleneglycol 6,000, titanium oxide, talc, iron oxide red and iron oxide yellow in ethanol and purified water, in conventional tablet coating conditions.

### Example 5

### Press-Coated Tablet of Simvastatin-Aspirin

(1) Simvastatin Granules

According to the components and contents shown in Table 1 below, simvastatin granules were prepared in the same manner as in Example 1.

(2) Aspirin Granules

According to the components and contents shown in Table 1 below, aspirin granules were prepared in the same manner as in Example 2.

(3) Tableting and Coating

The aspirin granules of Example 5-(2) were compressed into tablets in a rotary compressor (MRC-30, Sejong Machinery Co., Korea). The prepared tablets were used as inner cores and compressed together with the simvastatin granules of Example 5-(1) in a rotary tablet press (RUD-1, Kilian), thus preparing press-coated tablets. The prepared press-coated tablets were coated with a coating solution, obtained by dissolving hydroxypropylmethylcellulose 2910, hydroxypropylcellulose, titanium oxide, talc, iron oxide red and iron oxide yellow in ethanol and purified water, in conventional tablet coating conditions.

### Example 6

### Press-Coated Tablet of Simvastatin-Aspirin

According to the components and contents shown in Table 1 below, press-coated tablets of simvastatin-aspirin were prepared in the same manner as in Example 5, except that the aspirin tablets were additionally coated with ethylcellulose, before they were compressed into the press-coated tablets.

### Example 7

### Press-Coated Tablet of Simvastatin-Aspirin

According to the components and contents shown in Table 1 below, press-coated tablets of simvastatin-aspirin were prepared in the same manner as in Example 5, except that hydroxypropylmethylcellulose phthalate was used instead of ethylcellulose.

### Example 8

### Press-Coated Tablet of Simvastatin-Aspirin

(1) Simvastatin Granules

According to the components and contents shown in Table 1 below, simvastatin, lactose and microcrystalline cellulose were sieved through sieve No. 20, and then mixed with each other for 20 minutes. The mixture was placed in a high-shear mixer, in which it was kneaded with a binding solution of hydroxypropylcellulose dissolved in purified water. After completion of the kneading process, the kneaded material was dried and sieved, and then butylhydroxyanisol and stearic acid, sieved through sieve No. 35, were added to and mixed with the dried material for 4 minutes.

(2) Aspirin Granules

According to the components and contents shown in Table 1 below, aspirin granules were prepared in the same manner as in Example 2.

(3) Tableting and Coating

The simvastatin granules of Example 8-(1) were compressed into tablets in a rotary compressor (MRC-30, Sejong Machinery Co., Korea). The prepared tablets were used as inner cores and compressed together with the aspirin granules of Example 8-(2) in a rotary tablet press (RUD-1, Kilian), thus preparing press-coated tablets. The prepared press-coated tablets were coated with a coating solution, obtained by dissolving hydroxypropylmethylcellulose 2910, hydroxypropylcellulose, titanium oxide, talc, iron oxide red and iron oxide yellow in ethanol and purified water, in conventional tablet coating conditions.

### Example 9

### Press-Coated Tablet of Simvastatin-Aspirin

According to the components and contents shown in Table 2 below, press-coated tablets of simvastatin-aspirin were prepared in the same manner as in Example 8, except that the simvastatin tablets were additionally coated with hydroxypropylcellulose phthalate, before they were compressed into the press-coated tablets.

### Example 10 to 13

### Press-Coated Tablets of Simvastatin-Aspirin

According to the components and contents shown in Table 2 below, press-coated tablets of simvastatin-aspirin were prepared in the same manner as in Example 9, except that the amount of use of aspirin was changed.

### Example 14

### Multilayer Tablet (Bilayer Tablet) of Atorvastatin-Aspirin

(1) Atorvastatin Granules

According to the components and contents shown in Table 2 below, atorvastatin granules were prepared in the same manner as in Example 3-(1).

(2) Aspirin Granules

According to the components and contents shown in Table 2 below, aspirin and talc were mixed with each other in a double cone mixer for 5 minutes to prepare a mixture. Meanwhile, hydroxypropylmethylcellulose (6 cps) and PEG 6,000 were dissolved in an ethanol-methylene chloride solution to prepare a first coating solution. The mixture was coated with the first coating solution, and then coated with a second coating solution, obtained by dissolving ethylcellulose and triethyl citrate in an ethanol-methylene chloride solution. After completion of the coating process, the coated material was mixed with microcrystalline cellulose, pre-gelatinized starch and colloidal silicon dioxide in a double cone mixer for 20 minutes. Then, stearic acid sieved through sieve No. 35 was added to and mixed with the mixture for 4 minutes, thus preparing aspirin granules.

(3) Tableting and Coating

The atorvastatin granules of Example 14-(1) and the aspirin granules of Example 14-(2) were fed into the hoppers of a multilayer compressor (MRC-37, Sejong Machinery Co., Korea), in which the granules were compressed into bilayer tablets. The prepared tablets were coated with a coating solution, obtained by dissolving hydroxypropylmethylcellulose 2910, hydroxypropylcellulose, titanium oxide, talc, iron oxide yellow and iron oxide red in ethanol and purified water, in conventional tablet coating conditions.

### Example 15

### Multilayer Tablet (Trilayer Tablet) of Atorvastatin-Aspirin

(1) Atorvastatin Granules

According to the components and contents shown in Table 2 below, atorvastatin granules were prepared in the same manner as in Example 3-(1).

(2) Aspirin Granules

According to the components and contents shown in Table 2 below, aspirin granules were prepared in the same manner as in Example 14-(2).

(3) Placebo Granules

According to the components and contents shown in Table 2 below, microcrystalline cellulose, lactose, Copovidone and sodium stearyl fumarate were mixed with each other for 20 minutes to placebo granules.

(4) Tableting and Coating

The atorvastatin granules of Example 15-(1), the aspirin granules of Example 15-(2) and the placebo granules of Example 15-(3) were fed into the respective hoppers of a multilayer compressor (MRC-37, Sejong Machinery Co., Korea), in which the granules were compressed into trilayer tablets. Herein, the placebo granules were fed into No. 2 hopper, such that they were disposed in a middle layer. The prepared tablets were coated with a coating solution, obtained by dissolving hydroxypropylmethylcellulose 2910, hydroxypropylcellulose, titanium oxide, talc, iron oxide yellow and iron oxide red in ethanol and purified water, in conventional tablet coating conditions.

### Example 16

### Multilayer Tablet (Bilayer Tablet of Atorvastatin-Aspirin

According to the components and contents shown in Table 2 below, bilayer tablets of atorvastatin-aspirin were prepared in the same manner as in Example 14, except that the aspirin granules of Example 14-(2) were mixed with aspirin, alginic acid, hydroxypropylmethylcellulose, pre-gelatinized starch, microcrystalline cellulose and colloidal silicon dioxide in a double cone mixer for 20 minutes, and then, stearic acid sieved through sieve No. 35 was added to and mixed with the mixture for 4 minutes.

### Example 17

### Multilayer Tablet (Bilayer Tablet) of Simvastatin-Aspirin

(1) Simvastatin Granules

According to the components and contents shown in Table 3 below, simvastatin, microcrystalline cellulose and lactose were mixed with each other. Then, the mixture was placed in a high-shear mixer, in which it was kneaded with a binding solution, obtained by dissolving hydroxypropylcellulose in a purified water. The kneaded material was dried in a drying oven, and then mixed with butylhydroxyanisol, sodium alginate and hydroxypropylmethylcellulose for 20 minutes. Then, stearic acid sieved through sieve No. 35 was added to and mixed with the mixture for 4 minutes, thus preparing simvastatin granules.

(2) Aspirin Granules

According to the components and contents shown in Table 3 below, aspirin granules were prepared in the same manner as in Example 2.

(3) Tableting and Coating

The simvastatin granules of Example 17-(1) and the aspirin granules of Example 17-(2) were fed into the respective hoppers of a multilayer compressor (MRC-37, Sejong Machinery Co., Korea), in which the granules were compressed into bilayer tablets. The obtained tablets were coated with a coating solution, obtained by dissolving hydroxypropylmethylcellulose 2910, hydroxypropylcellulose, titanium oxide, talc, iron oxide yellow and iron oxide red in ethanol and purified water, in conventional tablet coating conditions.

### Example 18

### Multilayer Tablet (Bilayer Tablet) of Simvastatin-Aspirin

According to the components and contents shown in Table 3 below, bilayer tablets of simvastatin-aspirin were prepared in the same manner as in Example 17, except that the aspirin granules were mixed with aspirin, microcrystalline cellulose, Crosspovidone, pre-gelatinized starch and colloidal silicon dioxide for 20 minutes, and then stearic acid sieved through sieve No. 35 was mixed with the mixture for 4 minutes.

### Example 19

### Multilayer Tablet (Bilayer Tablet of Simvastatin-Aspirin

According to the components and contents shown in Table 3 below, bilayer tablets of simvastatin-aspirin were prepared in the same manner as in Example 17, except that the amount of simvastatin in the simvastatin granules was increased, the aspirin granules were mixed with aspirin, microcrystalline cellulose, crosspovidone, pre-gelatinized starch and colloidal silicon dioxide for 20 minutes, and then stearic acid sieved through sieve No. 35 was mixed with the mixture for 4 minutes.

### Example 20

### Multilayer Tablet (Bilayer Tablet) of Simvastatin-Aspirin

According to the components and contents shown in Table 3 below, bilayer tablets of simvastatin-aspirin were prepared in the same manner as in Example 17, except for the following. The aspirin granules were mixed with microcrystalline cellulose, magnesium oxide, magnesium carbonate and calcium carbonate for 20 minutes, and the mixture was kneaded with a binding solution, obtained by dissolving hydroxypropylcellulose in purified water, and was dried. The dried material was mixed with aspirin, pre-gelatinized starch and sodium starch glycolate for 10 minutes, and then mixed with stearic acid, sieved through sieve No. 35, for 4 minutes.

### Example 21

### Multilayer Tablet (Trilayer Tablet) of Simvastatin-Aspirin

(1) Simvastatin Granules

According to the components and contents shown in Table 3 below, simvastatin granules were prepared in the same manner as in Example 17-(1).

(2) Aspirin Granules

According to the components and contents shown in Table 3 below, the aspirin granules were prepared in the same manner as in Example 17-(2).

(3) Placebo Granules

According to the components and contents shown in Table 3 below, microcrystalline cellulose, lactose, Copovidone and sodium stearyl fumarate were mixed with each other for 20 minutes to prepare placebo granules.

(4) Tableting and Coating

The atorvastatin granules of Example 21-(1), the aspirin granules of Example 21-(2) and the placebo granules of Example 21-(3) were fed into the respective hoppers of a multilayer compressor (MRC-37, Sejong Machinery Co., Korea), in which the granules were compressed into trilayer tablets. Herein, the placebo granules were fed into No. 2 hopper, such that they were disposed in a middle layer. The prepared tablets were coated with a coating solution, obtained by dissolving hydroxypropylmethylcellulose 2910, hydroxypropylcellulose, titanium oxide, talc, iron oxide yellow and iron oxide red in ethanol and purified water, in conventional tablet coating conditions.

### Example 22

### Simvastatin-Aspirin Capsule Formulation (Granule+Granule)

(1) Simvastatin Granules

According to the components and contents shown in Table 3 below, simvastatin, lactose and microcrystalline cellulose were mixed with each other in a double cone mixer for 20 minutes to prepare a mixture. Meanwhile, hydroxypropylcellulose was dissolved in purified water to prepare a binding solution. The mixture was placed in a high-shear mixer, in which it was kneaded with the binding solution and dried. After completion of the drying process, the dried material was mixed with butylhydroxyanisol and stearic acid for 4 minutes, thus preparing simvastatin granules.

(2) Aspirin Granules

According to the components and contents shown in Table 3 below, aspirin granules were prepared in the same manner as in Example 1.

(3) Filling

The simvastatin granules of Example 22-(1) and the aspirin granules of Example 22-(2) were filled in capsule No. 1, thus preparing capsule formulations.

### Example 23

### Simvastatin-Aspirin Capsule Formulation (Granule+Tablet)

(1) Simvastatin Granules

According to the components and contents shown in Table 3 below, simvastatin granules were prepared in the same manner as in Example 22.

(2) Aspirin Tablets

According to the components and contents shown in Table 3 below, aspirin, microcrystalline cellulose, pre-gelatinized starch, carbomer and colloidal silicon dioxide were mixed with each other for 20 minutes, and then mixed with stearic acid, sieved through sieve No. 35, for 4 minutes, thus preparing granules. The granules were compressed into tablets in a rotary compressor (MRC 3—: Sejong Machinery Co., Korea). The tablets were coated with a coating solution, obtained by dissolving ethylcellulose and triethyl citrate in ethanol, thus preparing aspirin tablets.

(3) Filling

The simvastatin granules of Example 23-(1) and the aspirin tablets of Example 23-(2) were filled in capsule No. 1, thus preparing capsule formulations.

### Example 24

### Simvastatin+Aspirin Capsule Formulation (Granule+Pellet)

(1) Simvastatin Granules

According to the components and contents shown in Table 3 below, simvastatin granules were prepared in the same manner as in Example 22.

(2) Preparation of Aspirin Pellets

According to the components and contents shown in Table 3 below, sugar spheres, aspirin and microcrystalline cellulose were placed in a centrifugal fluidizing coating granulator (Freund), into which a binding solution, obtained by dissolving hydroxypropylcellulose in ethanol, and aspirin, were supplied, thus preparing spherical granules. The spherical granules were coated with a solution of ethylcellulose and triethyl citrate in ethanol, thus preparing aspirin pellets.

(3) Filling in Capsule

The simvastatin granules of Example 24-(1) and the aspirin pellets of Example 24-(2) were filled in a capsule, thus preparing capsule formulations.

### Example 25

### Atorvastatin-Aspirin Capsule Formulation (Granule+Tablet)

According to the components and contents shown in Table 4 below, an atorvastatin-aspirin capsule formulation was prepared in the same manner as in Example 23, except that the atorvastatin granules of Example 3 were used instead of the simvastatin granules.

### Example 26

### Fluvastatin-Aspirin Capsule Formulation (Granule+Tablet)

According to the components and contents shown in Table 4 below, a fluvastatin-aspirin capsule formulation was prepared in the same manner as in Example 23, except that fluvastatin was used instead of simvastatin.

### Example 27

### Lovastatin-Aspirin Capsule Formulation (Granule+Tablet)

According to the components and contents shown in Table 4 below, a lovastatin-aspirin capsule formulation (granule+tablet) was prepared in the same manner as in Example 23, except that lovastatin was used instead of simvastatin.

### Example 28

### Rosuvastatin-Aspirin Capsule Formulation (Granule+Tablet)

According to the components and contents shown in Table 4 below, a rosuvastatin-aspirin capsule formulation (granule+tablet) was prepared in the same manner as in Example 23, except that rosuvastatin was used instead of simvastatin.

### Example 29

### Pravastatin-Aspirin Capsule Formulation (Granule+Tablet)

According to the components and contents shown in Table 4 below, a pravastatin-aspirin capsule formulation (granule+tablet) was prepared in the same manner as in Example 23, except that pravastatin was used instead of simvastatin.

### Example 30

### Pitavastatin-Aspirin Capsule Formulation (Granule+Tablet)

According to the components and contents shown in Table 4 below, a pitavastatin-aspirin capsule formulation (granule+tablet) was prepared in the same manner as in Example 23, except that pitavastatin was used instead of simvastatin.

### Example 31

### Cerivastatin-Aspirin Capsule Formulation (Granule+Tablet)

According to the components and contents shown in Table 4 below, a cerivastatin-aspirin capsule formulation (granule+tablet) was prepared in the same manner as in Example 23, except that cerivastatin was used instead of simvastatin.

### Example 32

### Simvastatin-Aspirin Capsule Formulation (Tablet+Granule)

(1) Simvastatin Tablets

According to the components and contents shown in Table 4 below, simvastatin, lactose and microcrystalline cellulose were mixed with each other in a double cone mixer for 20 minutes to prepare a mixture. Meanwhile, hydroxypropylcellulose was dissolved in purified water to prepare a binding solution. The mixture was placed in a high-shear mixer, in which it was kneaded with the binding solution. The kneaded material was dried. After completion of the drying process, the dried material was mixed with butylhydroxyanisol and stearic acid for 4 minutes and compressed into tablets in a rotary compressor (MRC 30: Sejong Machinery Co., Korea). The obtained tablets were coated with a solution of hydroxypropylmethylcellulose and polyethyleneglycol in ethanol, thus preparing simvastatin tablets.

(2) Aspirin Granules

According to the components and contents shown in Table 4 below, aspirin, microcrystalline cellulose, pre-gelatinized starch and colloidal silicon dioxide were mixed with each other for 20 minutes. Then, stearic acid, sieved through sieve No. 35, was added to and mixed with the mixture for 4 minutes, thus preparing aspirin granules.

(3) Filling

The simvastatin tablets of Example 32-(1) and the aspirin granules of Example 32-(2) were filled in capsule No. 1, thus preparing capsule formulations.

### Example 33

### Simvastatin+Aspirin Capsule Formulation (Tablet+Tablet)

(1) Simvastatin Tablets

According to the components and contents shown in Table 5 below, simvastatin tablets were prepared in the same manner as in Example 32.

(2) Aspirin Tablets

According to the components and contents shown in Table 5 below, aspirin, microcrystalline cellulose, pre-gelatinized starch and colloidal silicon dioxide were mixed with each other in a mixer for 20 minutes to prepare a mixture. Then, stearic acid, sieved through sieve No. 35, was added to and mixed with the mixture for 4 minutes to prepare aspirin granules. The aspirin granules were compressed into tablets in a rotary compressor (MRC 30, Sejong Machinery Co., Korea), and the tablets were coated with a solution of hydroxypropylmethylcellulose and polyethyleneglycol in ethanol, thus preparing aspirin tablets.

(3) Filling

The simvastatin tablets of Example 33-(1) and the aspirin tablets of Example 33-(2) were filled in capsule No. 0, thus preparing capsule formulations.

### Example 34

### Simvastatin+Aspirin Capsule Formulation (Tablet+Pellet)

(1) Simvastatin Tablets

According to the components and contents shown in Table 5 below, a simvastatin tablet+aspirin capsule formulation was prepared in the same manner as in Example 32.

(2) Aspirin Pellets

According to the components and contents shown in Table 5 below, sugar spheres, aspirin and lactose were placed in a centrifugal fluidizing coating granulator (Freund), into which a binding solution, obtained by dissolving hydroxypropylcellulose in ethanol, and aspirin, were supplied, thus preparing spherical granules. The spherical granules were coated with a solution of hydroxypropylmethylcellulose and polyethyleneglycol 6,000 in ethanol, thus preparing aspirin pellets.

(3) Filling

The simvastatin tablets of Example 34-(1) and the aspirin pellets of Example 34-(2) were filled in capsule No. 0, thus preparing capsule formulations.

### Example 35

### Simvastatin-Aspirin Capsule Formulation (Pellet+Granule)

(1) Simvastatin Pellets

According to the components and contents shown in Table 5 below, sugar spheres, simvastatin and lactose were placed in a centrifugal fluidizing coating granulator (Freund), into which a binding solution, obtained by dissolving hydroxypropylcellulose butylhydroxyanisol in ethanol, together with simvastatin and lactose, were supplied, thus preparing spherical granules. The spherical granules were coated with a solution of hydroxypropylmethylcellulose and polyethyleneglycol 6,000 in ethanol, thus preparing simvastatin pellets.

(2) Aspirin Granules

According to the components and contents shown in Table 5 below, aspirin granules were prepared in the same manner as in Example 22.

(3) Filling

The simvastatin pellets of Example 35-(1) and the aspirin granules of Example 35-(2) were filled in No. 1 capsule, thus preparing capsule formulations.

### Example 36

### Simvastatin-Aspirin Capsule Formulation (Pellet+Tablet)

(1) Simvastatin Pellets

According to the components and contents shown in Table 5 below, simvastatin pellets were prepared in the same manner as in Example 35.

(2) Aspirin Tablets

According to the components and contents shown in Table 5 below, aspirin tablets were prepared in the same manner as in Example 23, except that the amount of use of aspirin was changed.

(3) Filling

The simvastatin pellets of Example 36-(1) and the aspirin tablets of Example 36-(2) were filled in capsule No. 1, thus preparing capsule formulations.

### Example 37

### Simvastatin-Aspirin Capsule Formulation (Pellet+Pellet)

(1) Simvastatin Pellets

According to the components and contents shown in Table 5 below, simvastatin pellets were prepared in the same manner as in Example 35.

(2) Aspirin Pellets

According to the components and contents shown in Table 5 below, aspirin pellets were prepared in the same manner as in Example 24.

(3) Filling

The simvastatin pellets of Example 37-(1) and the aspirin pellets of Example 37-(2) were filled in capsule No. 0, thus preparing capsule formulations.

### Example 38

### Atorvastatin+Aspirin Capsule Formulation (Mini-Tablet+Mini-Tablet)

(1) Atorvastatin Mini-Tablets

According to the components and contents shown in Table 5 below, the atorvastatin granules of Example 3 were compressed into 2.5 mm mini-tablets in a rotary compressor (Killian).

(2) Aspirin Mini-Tablets

According to the components and contents shown in Table 5 below, the atorvastatin granules of Example 2 were compressed into 2.5-mm mini-tablets in a rotary compressor (Killian). The obtained tablets were placed in a fluidized bed coating machine, in which they were coated with a solution of hydroxypropylcellulose phthalate and acetylated monoglyceride in an ethanol-methylene chloride mixture.

(3) Filling

The atorvastatin mini-tablets of Example 38-(1) and the aspirin mini-tablets of Example 38-(2) were filled in capsule No. 0, thus preparing capsule formulation.

### Example 39

### Simvastatin+Aspirin Kit

According to the components and contents shown in Table 5 below, simvastatin+aspirin kits were prepared in the same manner as in Example 33, except that, instead of filling the simvastatin tablet and the aspirin tablet in the capsule, the simvastatin tablet and the aspirin tablet were packaged in a blister package such that the tablets could be simultaneously administered.

### Example 40

### Atorvastatin+Aspirin Kit

(1) Atorvastatin Tablets

According to the components and contents shown in Table 5 below, the atorvastatin granules of Example 3 were compressed into tablets in a rotary compressor. The obtained tablets were coated with a solution of Opadry/acryl-eze (93F 19225, Colorcon) in purified water, thus preparing atorvastatin tablets.

(2) Aspirin Tablets

According to the components and contents shown in Table 5 below, the aspirin granules of Example 2 were compressed into tablets in a rotary compressor. The obtained tablets were coated with a solution of hydroxypropylcellulose and polyethyleneglycol 6,000 in ethanol-methylene chloride, thus preparing aspirin tablets.

(3) Packaging

The atorvastatin tablets of Example 40-(1) and the aspirin tablets of Example 40-(2) were packaged in a blister package, such that they could be simultaneously administered.

TABLE 1

| | | | Composition ratio | | | | | | | |
|---|---|---|---|---|---|---|---|---|---|---|
| | Function | Component | Example 1 | Example 2 | Example 3 | Example 4 | Example 5 | Example 6 | Example 7 | Example 8 |
| HMG | Active | Simvastatin | 20.0 | 20.0 | — | 80.0 | 20.0 | 20.0 | 20.0 | 20.0 |
| CoA | ingredient | | | | | | | | | |
| reductase | Active | Atorvastatin | — | — | 21.69 | — | — | — | — | — |
| inhibitor | ingredient | calcium | | | | | | | | |
| layer | Diluent | Lactose | 132.96 | 102.96 | — | — | 232.96 | 232.96 | 232.96 | 32.96 |
| | Diluent | Microcrystalline | 100.00 | 80.00 | 48.31 | — | 150.00 | 150.00 | 150.00 | 20.00 |
| | | cellulose | | | | | | | | |
| | Diluent | D- | — | — | 112.46 | — | — | — | — | — |
| | | mannitol | | | | | | | | |
| | | D- | | | | | | | | |
| | | mannitol | | | | | | | | |
| | Binder | Hydroxy | 9.00 | 4.00 | 5.00 | — | 14.00 | 14.00 | 14.00 | 4.00 |
| | | propylmethyl | | | | | | | | |
| | | cellulose | | | | | | | | |
| | Coating | Hydroxy | — | — | — | 15.96 | — | — | — | — |
| | base | propylmethyl | | | | | | | | |
| | | cellulose | | | | | | | | |
| | Disintegrant | Crospovidone | 5.00 | — | — | — | 10.00 | 10.00 | 10.00 | — |
| | Disintegrant | Sodium | — | — | 1.00 | — | — | — | — | — |
| | | starch | | | | | | | | |
| | | glycolate | | | | | | | | |
| | Acidifying | Citric | 1.00 | 1.00 | 2.00 | — | 1.00 | 1.00 | 1.00 | — |
| | agent | acid | | | | | | | | |
| | Stabilizer | Butylhydroxyanisol | 0.04 | 0.04 | 0.04 | 0.04 | 0.04 | 0.04 | 0.04 | 0.04 |
| | glidant | Colloidal | — | — | 1.00 | — | — | — | — | — |
| | | silicon | | | | | | | | |
| | | dioxide | | | | | | | | |
| | Lubricant | Talc | — | — | — | 2.00 | — | — | — | — |
| | Lubricant | Stearic | 1.00 | 1.00 | 1.50 | — | 6.00 | 6.00 | 6.00 | 1.00 |
| | | acid | | | | | | | | |
| | Coating | Eudragit | — | 30.00 | — | — | — | — | — | — |
| | base | L100-55 | | | | | | | | |
| | Plasticizer | Polyethyleneglycol | — | — | — | 2.00 | — | — | — | — |
| | | 6,000 | | | | | | | | |
| | Total amount | | 269.0 | 239.0 | 193.0 | 100.0 | 434.0 | 434.0 | 434.0 | 78.00 |
| Aspirin | Active | Aspirin | 40.00 | 40.00 | 40.00 | 40.00 | 40.00 | 40.00 | 40.00 | 40.00 |
| layer | ingredient | | | | | | | | | |
| | Diluent | Microcrystalline | 26.00 | 50.00 | 26.00 | 50.00 | 25.00 | 25.00 | 25.00 | 250.00 |
| | | cellulose | | | | | | | | |
| | Diluent | Pre- | 13.00 | 18.00 | 13.00 | 20.00 | 10.00 | 10.00 | 10.00 | 180.00 |
| | | gelatinized | | | | | | | | |
| | | starch | | | | | | | | |
| | Coating | Hydroxy | 4.00 | — | 4.00 | — | — | — | — | — |
| | base | propylmethyl | | | | | | | | |
| | | cellulose | | | | | | | | |
| | | (6 cps) | | | | | | | | |
| | Binder | Hydroxy | — | — | — | 10.00 | — | — | — | — |
| | | propylmethyl | | | | | | | | |
| | | cellulose | | | | | | | | |
| | | (100,000 cps) | | | | | | | | |
| | Binder | Copovidon | 5.00 | — | 5.00 | — | — | — | — | — |
| | Disintegrant | Alginic | — | — | — | 7.50 | — | — | — | — |
| | | acid | | | | | | | | |
| | glidant | Colloidal | 1.00 | 1.00 | 1.00 | 0.50 | 0.50 | 0.50 | 0.50 | 2.00 |
| | | silicon | | | | | | | | |
| | | dioxide | | | | | | | | |
| | Lubricant | Talc | 4.00 | — | 4.00 | — | — | — | — | — |
| | Plasticizer | Polyethyleneglycol | 0.40 | — | 0.40 | — | — | — | — | — |
| | | 6,000 | | | | | | | | |
| | Lubricant | Stearic | 1.00 | 1.00 | 1.00 | 1.00 | 0.50 | 0.50 | 0.50 | 2.00 |
| | | acid | | | | | | | | |
| | Coating | Methylcellulose | — | — | — | — | — | 10.00 | — | — |
| | base | | | | | | | | | |
| | Coating | Eudragit | 30.00 | — | 30.00 | — | — | — | — | — |
| | base | L100-55 | | | | | | | | |
| | Coating | Hydroxy | — | — | — | — | — | — | 10.00 | — |
| | base | propylmethyl | | | | | | | | |
| | | cellulose | | | | | | | | |
| | | phthalate | | | | | | | | |
| | Total amount | | 124.4 | 110.0 | 124.4 | 129.0 | 76.0 | 86.0 | 86.0 | 474.0 |
| Film | Film | Hydroxy | 4.00 | 4.00 | 4.00 | 4.00 | 16.00 | 16.00 | 16.00 | 16.00 |
| layer | coating | propylmethyl | | | | | | | | |
| | base | cellulose | | | | | | | | |
| | Film | Hydroxy | 4.00 | 4.00 | 4.00 | 4.00 | 5.00 | 5.00 | 5.00 | 5.00 |
| | coating | propyl | | | | | | | | |
| | base | cellulose | | | | | | | | |
| | Colorant | Titanium | 1.50 | 1.50 | 1.50 | 1.50 | 1.50 | 1.50 | 1.50 | 1.50 |
| | | oxide | | | | | | | | |
| | Dusting | Talc | 0.50 | 0.50 | 0.50 | 0.50 | 0.50 | 0.50 | 0.50 | 0.50 |
| | agent | | | | | | | | | |
| | Pigment | Iron | qs | qs | qs | qs | qs | qs | qs | qs |
| | | oxide | | | | | | | | |
| | | yellow | | | | | | | | |
| | Pigment | Iron | qs | qs | qs | qs | qs | qs | qs | qs |
| | | oxide | | | | | | | | |
| | | red | | | | | | | | |

| | | | | | | | | |
|---|---|---|---|---|---|---|---|---|
| Total amount | 10.0 | 10.0 | 10.0 | 10.0 | 23.0 | 23.0 | 23.0 | 23.0 |

| | | | | | | | | |
|---|---|---|---|---|---|---|---|---|
| Total amount | 403.4 | 359.0 | 327.4 | 239.0 | 533.0 | 543.0 | 543.0 | 575.0 |

TABLE 2

| | | | Composition ratio | | | | | | | |
|---|---|---|---|---|---|---|---|---|---|---|
| | | | | Example | Example | Example | Example | Example | Example | Example |
| | Function | Component | Example 9 | 10 | 11 | 12 | 13 | 14 | 15 | 16 |
| HMG | Active | Simvastatin | 20.0 | 20.0 | 20.0 | 20.0 | 20.0 | — | — | — |
| CoA | ingredient | | | | | | | | | |
| reductase | Active | Atorvastatin | — | — | — | — | — | 21.69 | 21.69 | 21.69 |
| inhibitor | ingredient | calcium | | | | | | | | |
| layer | Diluent | Lactose | 32.96 | 32.96 | 32.96 | 32.96 | 32.96 | — | — | — |
| | Diluent | Microcrystalline | 20.0 | 20.0 | 20.0 | 20.0 | 20.0 | 48.31 | 48.31 | 48.31 |
| | | cellulose | | | | | | | | |
| | Diluent | D- | — | — | — | — | — | 112.46 | 112.46 | 112.46 |
| | | mannitol | | | | | | | | |
| | | D- | | | | | | | | |
| | | mannitol | | | | | | | | |
| | Binder | Hydroxypropyl | 4.00 | 4.00 | 4.00 | 4.00 | 4.00 | 5.00 | 5.00 | 5.00 |
| | | cellulose | | | | | | | | |
| | Coating | Hydroxypropyl | 10.00 | 10.00 | 10.00 | 10.00 | 10.00 | — | — | — |
| | base | methyl | | | | | | | | |
| | | cellulose | | | | | | | | |
| | | phthalate | | | | | | | | |
| | Disintegrant | Sodium | — | — | — | — | — | 1.00 | 1.00 | 1.00 |
| | | starch | | | | | | | | |
| | | glycolate | | | | | | | | |
| | Acidifying | Citric | — | — | — | — | — | 2.00 | 2.00 | 2.00 |
| | agent | acid | | | | | | | | |
| | Stabilizer | Butylhydroxyanisol | 0.04 | 0.04 | 0.04 | 0.04 | 0.04 | 0.04 | 0.04 | 0.04 |
| | Glidant | Colloidal | — | — | — | — | — | 1.00 | 1.00 | 1.00 |
| | | silicon | | | | | | | | |
| | | dioxide | | | | | | | | |
| | Lubricant | Stearic | 1.00 | 1.00 | 1.00 | 1.00 | 1.00 | 1.50 | 1.50 | 1.50 |
| | | acid | | | | | | | | |
| | Total amount | | 88.0 | 88.0 | 88.0 | 88.0 | 88.0 | 193.0 | 193.0 | 193.0 |
| Aspirin | Active | Aspirin | 40.00 | 75.00 | 150.00 | 350.00 | 700.00 | 40.00 | 40.00 | 40.00 |
| layer | ingredient | | | | | | | | | |
| | Diluent | Microcrystalline | 250.00 | 250.00 | 200.00 | 100.00 | 100.00 | 50.00 | 50.00 | 50.00 |
| | | cellulose | | | | | | | | |
| | Diluent | Pre- | 180.00 | 180.00 | 150.00 | 75.00 | 75.00 | 25.60 | 25.60 | 20.00 |
| | | gelatinized | | | | | | | | |
| | | starch | | | | | | | | |
| | Coating | Hydroxyproyl | — | — | — | — | — | 4.00 | 4.00 | — |
| | base | methyl | | | | | | | | |
| | | cellulose | | | | | | | | |
| | | (6 cps) | | | | | | | | |
| | Binder | Hydroxypropyl | — | — | — | — | — | — | — | 10.00 |
| | | methyl | | | | | | | | |
| | | cellulose | | | | | | | | |
| | | (100,000 cps) | | | | | | | | |
| | Disintegrant | Alginic | — | — | — | — | — | — | — | 7.50 |
| | | acid | | | | | | | | |
| | Gelling | Colloidal | 2.00 | 2.00 | 2.00 | 4.00 | 4.00 | 1.00 | 1.00 | 0.50 |
| | agent | silicon | | | | | | | | |
| | | dioxide | | | | | | | | |
| | Lubricant | Talc | — | — | — | — | — | 4.00 | 4.00 | — |
| | Plasticizer | Polyethylene | — | — | — | — | — | 0.40 | 0.40 | — |
| | | glycol | | | | | | | | |
| | | 6,000 | | | | | | | | |
| | Plasticizer | Triethyl | — | — | — | — | — | 2.00 | 2.00 | — |
| | | citrate | | | | | | | | |
| | Lubricant | Stearic | 2.00 | 2.00 | 2.00 | 4.00 | 4.00 | 1.00 | 1.00 | 1.00 |
| | | acid | | | | | | | | |
| | Coating | Ethyl | — | — | — | — | — | 20.00 | 20.00 | — |
| | base | cellulose | | | | | | | | |
| | Total amount | | 474.0 | 509.0 | 504.0 | 533.0 | 883.0 | 148.0 | 148.0 | 129.0 |
| Placebo | Diluent | Microcrystalline | — | — | — | — | — | — | 83.0 | — |
| layer | | cellulose | | | | | | | | |
| | Diluent | Lactose | — | — | — | — | — | — | 20.0 | — |
| | Binder | Copovidone | — | — | — | — | — | — | 5.0 | — |
| | Lubricant | Sodium | — | — | — | — | — | — | 2.0 | — |
| | | stearyl | | | | | | | | |
| | | fumarate | | | | | | | | |
| | Total amount | | — | — | — | — | — | — | 110.0 | — |
| Film | Film | Hydroxypropyl | 16.00 | 16.00 | 16.00 | 16.00 | 20.00 | 4.00 | 10.00 | 4.00 |
| layer | coating | methyl | | | | | | | | |
| | base | cellulose | | | | | | | | |
| | Film | Hydroxypropyl | 5.00 | 5.00 | 5.00 | 5.00 | 5.00 | 4.00 | 4.00 | 4.00 |
| | coating | cellulose | | | | | | | | |
| | base | | | | | | | | | |
| | Colorant | Titanium | 1.50 | 1.50 | 1.50 | 1.50 | 1.50 | 1.50 | 1.50 | 1.50 |
| | | oxide | | | | | | | | |
| | Dusting | Talc | 0.50 | 0.50 | 0.50 | 0.50 | 0.50 | 0.50 | 0.50 | 0.50 |
| | agent | | | | | | | | | |
| | Pigment | Iron | qs | qs | qs | qs | qs | qs | qs | qs |
| | | oxide | | | | | | | | |
| | | yellow | | | | | | | | |
| | Pigment | Iron | qs | qs | qs | qs | qs | qs | qs | qs |
| | | oxide | | | | | | | | |
| | | red | | | | | | | | |

| | | | | | | | | |
|---|---|---|---|---|---|---|---|---|
| Total amount | 23.0 | 23.0 | 23.0 | 23.0 | 27.0 | 10.0 | 16.0 | 10.0 |

| | | | | | | | | |
|---|---|---|---|---|---|---|---|---|
| Total | 585.0 | 620.0 | 615.0 | 644.0 | 998.0 | 351.0 | 467.0 | 332.0 |

TABLE 3

| | | | Composition ratio | | | | | | | |
|---|---|---|---|---|---|---|---|---|---|---|
| | | | Example | Example | Example | Example | Example | Example | Example | Example |
| | Function | Component | 17 | 18 | 19 | 20 | 21 | 22 | 23 | 24 |
| HMG | Active | Simvastatin | 20.0 | 20.0 | 40.0 | 20.0 | 20.0 | 20.0 | 20.0 | 20.0 |
| CoA | ingredient | | | | | | | | | |
| reductase | Diluent | Lactose | 132.96 | 132.96 | 132.96 | 132.96 | 132.96 | 32.96 | 32.96 | 32.96 |
| inhibitor | Diluent | Microcrystalline | 100.00 | 100.00 | 100.00 | 100.00 | 100.00 | 20.00 | 20.00 | 20.00 |
| layer | | cellulose | | | | | | | | |
| | Binder | Hydroxypropyl | 9.00 | 9.00 | 9.00 | 9.00 | 9.00 | 4.00 | 4.00 | 4.00 |
| | | cellulose | | | | | | | | |
| | Binder | Hydroxypropyl | 20.00 | 20.00 | 20.00 | 20.00 | 20.00 | — | — | — |
| | | methyl | | | | | | | | |
| | | cellulose | | | | | | | | |
| | | (100,000 cps) | | | | | | | | |
| | Disintegrant | Sodium | 20.00 | 20.00 | 20.00 | 20.00 | 20.00 | — | — | — |
| | | alginate | | | | | | | | |
| | Stabilizer | Butylhydroxyanisol | 0.04 | 0.04 | 0.04 | 0.04 | 0.04 | 0.04 | 0.04 | 0.04 |
| | Lubricant | Stearic | 1.00 | 1.00 | 1.00 | 1.00 | 1.00 | 1.00 | 1.00 | 1.00 |
| | | acid | | | | | | | | |
| | Total amount | | 303.0 | 303.0 | 323.0 | 303.0 | 303.0 | 78.0 | 78.0 | 78.0 |
| Aspirin | Active | Aspirin | 40.00 | 40.00 | 75.00 | 40.00 | 40.00 | 40.00 | 75.00 | 40.00 |
| layer | ingredient | | | | | | | | | |
| | Diluent | Microcrystalline | 50.00 | 50.00 | 50.00 | 40.00 | 50.00 | 26.00 | 26.00 | 20.00 |
| | | cellulose | | | | | | | | |
| | Diluent | Pre- | 18.00 | 18.00 | 18.00 | 15.00 | 18.00 | 13.00 | 13.00 | — |
| | | gelatinized | | | | | | | | |
| | | starch | | | | | | | | |
| | Diluent | Sugar | — | — | — | — | — | — | — | 100.00 |
| | | sphere | | | | | | | | |
| | | (1000 μm) | | | | | | | | |
| | Diluent | Magnesium | — | — | 100.00 | — | — | — | — | — |
| | | hydrogen | | | | | | | | |
| | | carbonate | | | | | | | | |
| | Diluent | Magnesium | — | — | | 25.00 | — | — | — | — |
| | | oxide | | | | | | | | |
| | Diluent | Magnesium | — | — | — | 15.00 | — | — | — | — |
| | | carbonate | | | | | | | | |
| | Diluent | Carbomer | — | — | — | — | — | — | 10.00 | — |
| | | 71G | | | | | | | | |
| | Diluent | Calcium | — | — | — | 43.00 | — | — | — | — |
| | | carbonate | | | | | | | | |
| | Binder | Hydroxypropyl | — | — | — | 5.00 | — | — | — | 5.00 |
| | | cellulose | | | | | | | | |
| | Coating | Hydroxypropyl | — | — | — | — | — | 4.00 | — | — |
| | base | methylcellulose | | | | | | | | |
| | | (6 cps) | | | | | | | | |
| | Binder | Copovidon | — | — | — | — | — | 5.00 | — | — |
| | Disintegrant | Crosspovidon | — | 5.00 | 5.00 | — | — | — | — | — |
| | Disintegrant | Sodium | — | — | — | 10.00 | — | — | — | — |
| | | starch | | | | | | | | |
| | | glycolate | | | | | | | | |
| | glidant | Colloidal | 1.00 | 1.00 | 1.00 | — | 1.00 | 1.00 | 1.00 | — |
| | | silicon | | | | | | | | |
| | | dioxide | | | | | | | | |
| | Acidifying | Citric acid | — | — | 20.00 | — | — | — | — | — |
| | agent | | | | | | | | | |
| | Lubricant | Talc | — | — | — | — | — | 4.00 | — | — |
| | Plasticizer | Polyethylene | — | — | — | — | — | 0.40 | — | — |
| | | glycol | | | | | | | | |
| | | 6,000 | | | | | | | | |
| | Plasticizer | Triethyl | — | — | — | — | — | — | 1.00 | 1.00 |
| | | citrate | | | | | | | | |
| | Lubricant | Stearic | 1.00 | 1.00 | 1.00 | 1.00 | 1.00 | 1.00 | 1.00 | — |
| | | acid | | | | | | | | |
| | Coating | Eudragit | — | — | — | — | — | 30.00 | — | — |
| | base | L100-55 | | | | | | | | |
| | Coating | Ethylcellulose | — | — | — | — | — | — | 10.00 | 10.00 |
| | base | | | | | | | | | |
| | Total amount | | 110.0 | 115.0 | 270.0 | 194.0 | 110.0 | 124.4 | 137.0 | 176.0 |
| Placebo | Diluent | Microcrystalline | — | — | — | — | 83.0 | — | — | — |
| layer | | cellulose | | | | | | | | |
| | Diluent | Lactose | — | — | — | — | 20.0 | — | — | — |
| | Binder | Copovidon | — | — | — | — | 5.0 | — | — | — |
| | Lubricant | Sodium | — | — | — | — | 2.0 | — | — | — |
| | | stearyl | | | | | | | | |
| | | fumarate | | | | | | | | |
| | Total amount | | — | — | — | — | 110.0 | — | — | — |
| Film | Film | Hydroxypropyl | 10.00 | 10.00 | 10.00 | 10.00 | 10.00 | — | — | 3.00 |
| layer | coating | methyl | | | | | | | | |
| | base | cellulose | | | | | | | | |
| | Film | Hydroxypropyl | 4.00 | 4.00 | 4.00 | 4.00 | 4.00 | — | — | 1.50 |
| | coating | cellulose | | | | | | | | |
| | base | | | | | | | | | |
| | Colorant | Titanium | 1.50 | 1.50 | 1.50 | 1.50 | 1.50 | — | — | 0.50 |
| | | oxide | | | | | | | | |
| | Dusting | Talc | 0.50 | 0.50 | 0.50 | 0.50 | 0.50 | — | — | 0.50 |
| | agent | | | | | | | | | |
| | Pigment | Iron oxide | qs | qs | qs | qs | qs | — | — | qs |
| | | yellow | | | | | | | | |
| | Pigment | Iron oxide | qs | qs | qs | qs | qs | — | — | qs |
| | | red | | | | | | | | |

| | | | | | | | | |
|---|---|---|---|---|---|---|---|---|
| Total amount | 16.0 | 16.0 | 16.0 | 16.0 | 16.0 | — | — | 5.5 |

| | | | | | | | | |
|---|---|---|---|---|---|---|---|---|
| Total | 429.0 | 434.0 | 609.0 | 513.0 | 539.0 | 202.4 | 215.0 | 259.5 |

TABLE 4

| | | | Composition ratio | | | | | | | |
|---|---|---|---|---|---|---|---|---|---|---|
| | | | Example | Example | Example | Example | Example | Example | Example | Example |
| | Function | Components | 25 | 26 | 27 | 28 | 29 | 30 | 31 | 32 |
| HMG | Active | Simvastatin | — | — | — | — | — | — | — | 20.00 |
| CoA | ingredient | | | | | | | | | |
| reductase | Active | Atorvastatin | 21.69 | — | — | — | — | — | — | — |
| inhibitor | ingredient | | | | | | | | | |
| layer | Active | Fluvastatin | — | 21.06 | — | — | — | — | — | — |
| | ingredient | sodium | | | | | | | | |
| | Active | Lovastatin | — | — | 20.00 | — | — | — | — | — |
| | ingredient | | | | | | | | | |
| | Active | Rosuvastatin | — | — | — | 10.40 | — | — | — | — |
| | ingredient | calcium | | | | | | | | |
| | Active | Pravastatin | — | — | — | — | 10.00 | — | — | — |
| | ingredient | sodium | | | | | | | | |
| | Active | Pitavastatin | — | — | — | — | — | 2.00 | — | — |
| | ingredient | calcium | | | | | | | | |
| | Active | Cerivastatin | — | — | — | — | — | — | 0.80 | — |
| | ingredient | sodium | | | | | | | | |
| | Diluent | Lactose | — | 33.86 | 34.96 | 35.56 | 34.96 | 44.96 | 46.16 | 32.96 |
| | Diluent | Microcrystalline | 48.31 | 20.00 | 20.00 | 20.00 | 20.00 | 20.00 | 20.00 | 20.00 |
| | | cellulose | | | | | | | | |
| | Diluent | D- | 112.46 | — | — | — | — | — | — | — |
| | | mannitol | | | | | | | | |
| | Binder | Hydroxypropyl | 5.00 | 4.00 | 4.00 | 4.00 | 4.00 | 4.00 | 4.00 | 4.00 |
| | | cellulose | | | | | | | | |
| | Disintegrant | Sodium | 1.00 | — | — | — | — | — | — | — |
| | | starch | | | | | | | | |
| | | glycolate | | | | | | | | |
| | Acidifying | Citric | 2.00 | — | — | — | — | — | — | — |
| | agent | acid | | | | | | | | |
| | Stabilizer | Butylhydroxyanisol | 0.04 | 0.04 | 0.04 | 0.04 | 0.04 | 0.04 | 0.04 | 0.04 |
| | Glidant | Colloidal | 1.00 | — | — | — | — | — | — | — |
| | | silicon | | | | | | | | |
| | | dioxide | | | | | | | | |
| | Coating | Hydroxypropyl | — | — | — | — | — | — | — | 10.00 |
| | base | methyl | | | | | | | | |
| | | cellulose | | | | | | | | |
| | Plasticizer | Polyethylene | — | — | — | — | — | — | — | 1.00 |
| | | glycol | | | | | | | | |
| | | 6,000 | | | | | | | | |
| | Lubricant | Stearic | 1.50 | 1.00 | 1.00 | 1.00 | 1.00 | 1.00 | 1.00 | 1.00 |
| | | acid | | | | | | | | |
| | Total amount | | 193.0 | 80.0 | 80.0 | 71.0 | 70.0 | 72.0 | 72.0 | 89.0 |
| Aspirin | Active | Aspirin | 75.00 | 75.00 | 75.00 | 75.00 | 75.00 | 75.00 | 75.00 | 40.00 |
| layer | ingredient | | | | | | | | | |
| | Diluent | Microcrystalline | 26.00 | 26.00 | 26.00 | 26.00 | 26.00 | 26.00 | 26.00 | 50.00 |
| | | cellulose | | | | | | | | |
| | Diluent | Pre- | 13.00 | 13.00 | 13.00 | 13.00 | 13.00 | 13.00 | 13.00 | 18.00 |
| | | gelatinized | | | | | | | | |
| | | starch | | | | | | | | |
| | Diluent | Carbomer | 10.00 | 10.00 | 10.00 | 10.00 | 10.00 | 10.00 | 10.00 | — |
| | | 71G | | | | | | | | |
| | Glidant | Colloidal | 1.00 | 1.00 | 1.00 | 1.00 | 1.00 | 1.00 | 1.00 | 1.00 |
| | | silicon | | | | | | | | |
| | | dioxide | | | | | | | | |
| | Plasticizer | Triethyl | 1.00 | 1.00 | 1.00 | 1.00 | 1.00 | 1.00 | 1.00 | — |
| | | citrate | | | | | | | | |
| | Lubricant | Stearic | 1.00 | 1.00 | 1.00 | 1.00 | 1.00 | 1.00 | 1.00 | 1.00 |
| | | acid | | | | | | | | |
| | Coating | Ethyl | 10.00 | 10.00 | 10.00 | 10.00 | 10.00 | 10.00 | 10.00 | |
| | base | cellulose | | | | | | | | |

| | | | | | | | | |
|---|---|---|---|---|---|---|---|---|
| Total amount | 137.0 | 137.0 | 137.0 | 137.0 | 137.0 | 137.0 | 137.0 | 110.0 |

| | | | | | | | | |
|---|---|---|---|---|---|---|---|---|
| Total | 330.0 | 217.0 | 217.0 | 208.0 | 207.0 | 209.0 | 209.0 | 199.0 |

TABLE 5

| | | | Composition ratio | | | | | | | |
|---|---|---|---|---|---|---|---|---|---|---|
| | | | Example | Example | Example | Example | Example | Example | | |
| | Function | Component | 33 | 34 | 35 | 36 | 37 | 38 | Example 39 | Example 40 |
| HMG | Active | Simvastatin | 20.00 | 20.00 | 20.00 | 20.00 | 20.00 | — | 20.00 | — |
| CoA | ingredient | | | | | | | | | |
| reductase | Active | Atorvastatin | — | — | — | — | — | 21.69 | — | 21.69 |
| inhibitor | ingredient | calcium | | | | | | | | |
| layer | Diluent | Lactose | 32.96 | 32.96 | 16.56 | 16.56 | 16.56 | — | 32.96 | — |
| | Diluent | Microcrystalline | 20.00 | 20.00 | — | — | — | 48.31 | 20.00 | 22.31 |
| | | cellulose | | | | | | | | |
| | Diluent | Sugar | — | — | 70.00 | 70.00 | 70.00 | — | — | — |
| | | sphere | | | | | | | | |
| | | (1000 μm) | | | | | | | | |
| | Diluent | D- | — | — | — | — | — | 112.46 | — | 50.46 |
| | | mannitol | | | | | | | | |
| | Binder | Hydroxypropyl | 4.00 | 4.00 | 4.00 | 4.00 | 4.00 | 5.00 | 4.00 | 3.50 |
| | | cellulose | | | | | | | | |
| | Coating | Hydroxypropyl | 10.00 | 10.00 | 4.00 | 4.00 | 4.00 | — | 10.00 | — |
| | base | methyl | | | | | | | | |
| | | cellulose | | | | | | | | |
| | | (6 cps) | | | | | | | | |
| | Coating | Acryleze | — | — | — | — | — | — | — | 10.50 |
| | base | 93F | | | | | | | | |
| | | 19225 | | | | | | | | |
| | Disintegrant | Sodium | — | — | — | — | — | 1.00 | — | 1.00 |
| | | starch | | | | | | | | |
| | | glycolate | | | | | | | | |
| | Acidifying | Citric | — | — | — | — | — | 2.00 | — | 1.50 |
| | agent | acid | | | | | | | | |
| | Stabilizer | Butylhydroxyaniosol | 0.04 | 0.04 | 0.04 | 0.04 | 0.04 | 0.04 | 0.04 | 0.04 |
| | glidant | Colloidal | — | — | — | — | — | 1.00 | — | 2.00 |
| | | silicon | | | | | | | | |
| | | dioxide | | | | | | | | |
| | Lubricant | Stearic | 1.00 | 1.00 | — | — | — | 1.50 | 1.00 | 1.00 |
| | | acid | | | | | | | | |
| | Plasticizer | Polyethylene | 1.00 | 1.00 | 0.40 | 0.40 | 0.40 | — | 1.00 | — |
| | | glycol | | | | | | | | |
| | | 6,000 | | | | | | | | |
| | Total amount | | 89.0 | 89.0 | 115.0 | 115.0 | 115.0 | 193.0 | 89.0 | 114.0 |
| Aspirin | Active | Aspirin | 40.00 | 40.00 | 40.00 | 40.00 | 40.00 | 40.00 | 40.00 | 40.00 |
| layer | ingredient | | | | | | | | | |
| | Dilutent | Microcrystalline | 50.00 | 20.00 | 26.00 | 26.00 | 20.00 | 50.00 | 50.00 | 50.00 |
| | | cellulose | | | | | | | | |
| | Dilutent | Pre- | 18.00 | — | 13.00 | 13.00 | — | 18.00 | 18.00 | 18.00 |
| | | gelatinized | | | | | | | | |
| | | starch | | | | | | | | |
| | Dilutent | Sugar | — | 100.00 | — | — | 100.00 | — | — | — |
| | | sphere | | | | | | | | |
| | | (1000 μm) | | | | | | | | |
| | Dilutent | Carbomer | — | — | — | 10.00 | — | — | — | — |
| | | 71G | | | | | | | | |
| | Coating | Hydroxypropyl | 4.00 | 5.00 | 4.00 | — | — | — | 4.00 | 5.50 |
| | base | methyl | | | | | | | | |
| | | cellulose | | | | | | | | |
| | | (6 cps) | | | | | | | | |
| | Binder | Hydroxypropyl | — | 5.00 | — | — | 5.00 | — | — | — |
| | | cellulose | | | | | | | | |
| | Binder | Copovidon | — | — | 5.00 | — | — | — | — | — |
| | glidant | Colloidal | 1.00 | — | 1.00 | 1.00 | — | 1.00 | 1.00 | 1.00 |
| | | silicon | | | | | | | | |
| | | dioxide | | | | | | | | |
| | Lubricant | Talc | — | — | 4.00 | — | — | — | — | — |
| | Plasticizer | Polyethylene | 0.40 | 0.50 | 0.40 | — | 0.50 | — | 0.40 | 0.50 |
| | | glycol | | | | | | | | |
| | | 6,000 | | | | | | | | |
| | Plasticizer | Triethyl | — | — | — | 1.00 | 1.00 | — | — | — |
| | | citrate | | | | | | | | |
| | Plasticizer | mivaset | — | — | — | — | — | 1.00 | — | — |
| | Lubricant | Stearic | 1.00 | — | 1.00 | 1.00 | — | 1.00 | 1.00 | 1.00 |
| | | acid | | | | | | | | |
| | Coating | Ethyl | — | — | — | 10.00 | 10.00 | — | — | — |
| | base | cellulose | | | | | | | | |
| | Coating | Eudragit | — | — | 30.00 | — | — | — | — | — |
| | base | L100- | | | | | | | | |
| | | 55 | | | | | | | | |
| | Coating | Hydroxypropyl | — | — | — | — | — | 9.00 | — | — |
| | base | methyl | | | | | | | | |
| | | cellulose | | | | | | | | |
| | | phthalate | | | | | | | | |

| | | | | | | | | |
|---|---|---|---|---|---|---|---|---|
| Total amount | 114.4 | 170.5 | 124.4 | 102.0 | 176.5 | 120.0 | 114.4 | 116.0 |

| | | | | | | | | |
|---|---|---|---|---|---|---|---|---|
| Total | 203.4 | 259.5 | 239.4 | 217.4 | 291.5 | 313.0 | 203.4 | 230.0 |

### Test Example 1

### Disintegration Test

The tablets obtained in Examples above were tested according to a disintegration method among general test methods described in the Korean Pharmacopoeia, eighth edition. In a specific test method, a disintegration tester was hung to a vertical axis member, placed in a beaker and moved upward and downward at 29-32 strokes per minute with amplitude of 53-57 mm. The movement of the tester was controlled such that, when the tester was moved down to the lowest position, the surface of a network disposed at the lower portion of the tester was 25 mm distant from the bottom of the beaker. Also, the amount of a test fluid in the beaker was adjusted such that, when the tester was moved down to the lowest position, the upper surface of the tester coincided with the surface of the fluid. The temperature of the fluid was maintained at 37±2° C. As the test fluid, a first fluid (pH: about 1.2) and a second fluid (pH: about 6.8) were used, and the test time was 2 hours for each of the test fluids.

6 samples for each Example were taken, each sample was placed in the glass tube of the tester, and the tester was immersed in the test fluid in the beaker, the temperature and fluid amount thereof have been controlled. The tester was moved upward and downward, and then the tester was carefully taken out of the test fluid, and the state of the sample in the glass tube was observed. The observation was performed at a 5-min interval up to 1 hour after the start of the test, and then at a 10-min interval. Because of the characteristics of the tablets, an auxiliary plate was not used. The test results are shown in Tables 6 and 7.

As can be seen from the results in Tables 6 and 7, the drugs of the bilayer tablets and trilayer tablets used in the tests disintegrated at time intervals intended in the present invention. Particularly, whether drugs are disintegrated absolutely influences gastric transit time and absorption. Particularly, in the case of preparations such as simvastatin, the pharmacokinetic parameters of which are difficult to predict only by a dissolution test due to their low solubility, the pharmacokinetic parameters can be determined by the disintegration test. It could be seen that, in the preparations of Examples 5, 6, 7, 14, 15 and 16, the HMG-CoA reductase inhibitor layer disintegrated faster as expected by the present inventors, and in the preparations of Examples 8, 9, 10, 11, 12, 13, 17, 18, 19, 20 and 21, the aspirin layer disintegrated faster. Also, it could be seen that the above disintegration rates were the same between simulated gastric fluid and simulated intestinal fluid. From the test results, it could be predicted that, when the preparations of Examples are administered orally, the HMG-CoA reductase inhibitor drug and the aspirin drug are dissolved and absorbed in vivo at different times. In addition, it can be seen that, even though the drugs are dissolved and absorbed in the same organ such as the stomach, they are dissolved and absorbed at different times. This suggests that, in the inventive combined pharmaceutical formulation of a HMG-CoA reductase inhibitor and aspirin, the decrease in the therapeutic effect of the drugs, caused by the interaction between the drugs, can be prevented.

TABLE 6

| | First fluid (pH 1.2) in disintegration test | | | |
|---|---|---|---|---|
| | HMG CoA reductase inhibitor | | Aspirin | |
| | Disinte- | | Disinte- | |
| | gration | Disintegration | gration | Disintegration |
| | time | rate* | time | rate |
| Example 5 | 21 min | Released faster | 38 min | Released slower |
| Example 6 | 18 min | Released faster | 55 min | Released slower |
| Example 7 | 23 min | Released faster | Not | Released slower |
| | | | inte- | |
| | | | grated | |
| Example 8 | 42 min | Released slower | 16 min | Released faster |
| Example 9 | Not | Released slower | 14 min | Released faster |
| | disinte- | | | |
| | grated | | | |
| Example 10 | Not | Released slower | 13 min | Released faster |
| | disinte- | | | |
| | grated | | | |
| Example 11 | Not | Released slower | 17 min | Released faster |
| | disinte- | | | |
| | grated | | | |
| Example 12 | Not | Released slower | 19 min | Released faster |
| | disinte- | | | |
| | grated | | | |
| Example 13 | Not | Released slower | 24 min | Released faster |
| | disinte- | | | |
| | grated | | | |
| Example 14 | 12 min | Released faster | 41 min | Released slower |
| Example 15 | 13 min | Released faster | 43 min | Released slower |
| Example 16 | 11 min | Released faster | 76 min | Released slower |
| Example 17 | 81 min | Released slower | 12 min | Released faster |
| Example 18 | 75 min | Released slower | 10 min | Released faster |
| Example 19 | 76 min | Released slower | 11 min | Released faster |
| Example 20 | 80 min | Released slower | 13 min | Released faster |
| Example 21 | 75 min | Released slower | 12 min | Released faster |

*Disintegration rate: in consideration of the disintegration times of the HMG CoA reductase inhibitor layer and the aspirin layer, the layer disintegrated faster was estimated as “released faster”, and the layer disintegrated slower “released slower”.

TABLE 7

| | Second fluid (pH 6.8) in disintegration test | | | |
|---|---|---|---|---|
| | HMG CoA reductase inhibitor | | Aspirin | |
| | Disinte- | | Disinte- | |
| | gration | Disintegration | gration | Disintegration |
| | time | rate* | time | rate |
| Example 5 | 21 min | Released faster | 38 min | Release slower |
| Example 6 | 18 min | Released faster | 55 min | Release slower |
| Example 7 | 23 min | Released faster | 45 min | Release slower |
| Example 8 | 42 min | Released slower | 16 min | Released faster |
| Example 9 | 41 min | Released slower | 14 min | Released faster |
| Example 10 | 38 min | Released slower | 13 min | Released faster |
| Example 11 | 42 min | Released slower | 17 min | Released faster |
| Example 12 | 45 min | Released slower | 19 min | Released faster |
| Example 13 | 47 min | Released slower | 24 min | Released faster |
| Example 14 | 12 min | Released faster | 41 min | Release slower |
| Example 15 | 13 min | Released faster | 43 min | Release slower |
| Example 16 | 11 min | Released faster | 76 min | Release slower |
| Example 17 | 81 min | Released slower | 12 min | Released faster |
| Example 18 | 75 min | Released slower | 10 min | Released faster |
| Example 19 | 76 min | Released slower | 11 min | Released faster |
| Example 20 | 80 min | Released slower | 13 min | Released faster |
| Example 21 | 75 min | Released slower | 12 min | Released faster |

### Test Example 2

### Dissolution Profile Test

The tablets obtained in Examples above were subjected to a dissolution test in conditions of stimulated gastric fluid (pH 1.2) or stimulated intestinal fluid (pH 6.8). However, in the case of simvastatin, a dissolution test described in the item “simvastatin tablet” of the United States Pharmacopoeia (USP 30) was carried out, because distinction was insufficient in the above conditions.

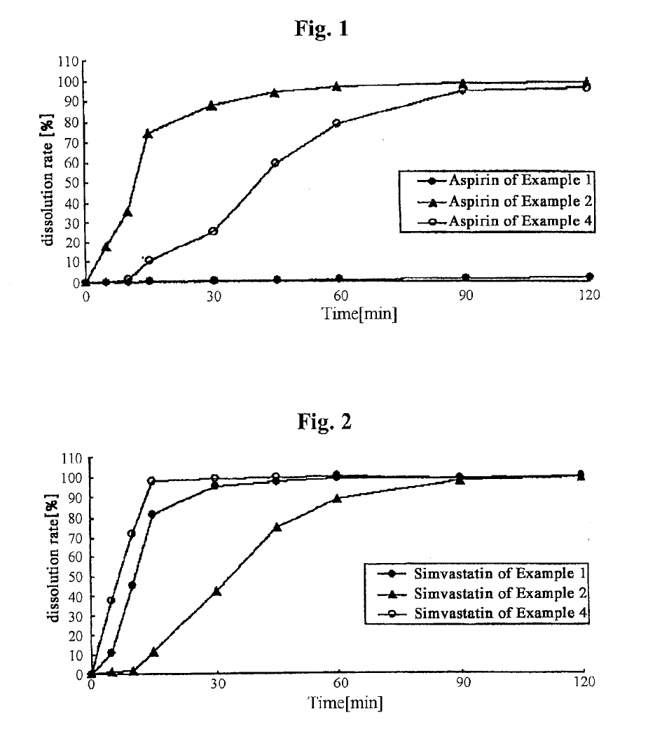

In a specific test method, 900 ml of stimulated gastric fluid (a first fluid in a disintegration test method described in the Korean Pharmacopoeia), which was heated to 37±0.5° C., was placed in a basket and a dissolution test was performed at a paddle rotation speed of 50 rpm. After 2 hours, a given amount of a sample for analysis was taken from the test fluid. Then, the test fluid was replaced with a simulated intestinal fluid (a second fluid in a disintegration test method described in the Korean Pharmacopoeia, 8^{th} edition), and a test was performed using the second test fluid. Six samples for each of Examples were taken, and one sample was placed in each basket. However, in the case of the capsule formulations, a sinker was used as an auxiliary device. After the start of dissolution, a given amount of dissolution medium was taken at a given interval of time and analyzed to measure dissolution rate. The measurement results are shown in FIGS. 1 to 7.

As shown in FIGS. 1 and 2, it was observed that, in the dissolution test carried out in the above test conditions, the inventive combined pharmaceutical formulations, comprising the HMG-CoA reductase inhibitor and aspirin, showed the difference in dissolution time between the two drug components. Particularly, it could be seen that, in the combined pharmaceutical formulation of Example 1, the simvastatin component was released faster, because aspirin was not substantially dissolved in the acidic pH range and was dissolved and released in the small intestines, whereas in the case of Example 2, aspirin was dissolved faster. In addition, it was found that, in the combined pharmaceutical formulation of Example 3, the reason why the dissolution of aspirin was delayed was because of the dissolution time of simvastatin coated on the surface of the combined pharmaceutical formulation, and the aspirin component could be absorbed in the stomach, whereas the simvastatin component could be absorbed quickly in the small intestines.

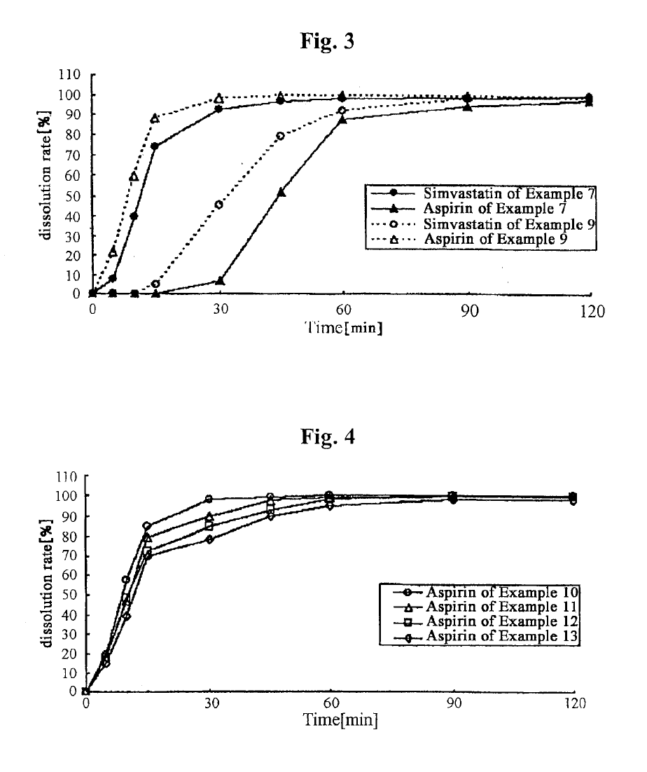

As shown in FIG. 3, it was observed that, in the press-coated tablet of Example 7, the dissolution of the aspirin component was delayed, because the simvastatin component was disposed in the outer layer, whereas in the press-coated tablet of Example 9, the dissolution of the simvastatin component was delayed, because the aspirin component was disposed in the outer layer. This suggests that, because the difference in dissolution time between the two drug components also occur in vivo, side effects, which can occur due to the simultaneous dissolution of the two drug components, can be reduced.

As can be seen in FIG. 4, the results of dissolution tests for the preparations of Examples 10, 11, 12 and 13, which had increased aspirin doses, revealed that there was little or no change in dissolution rate according to aspirin dose.

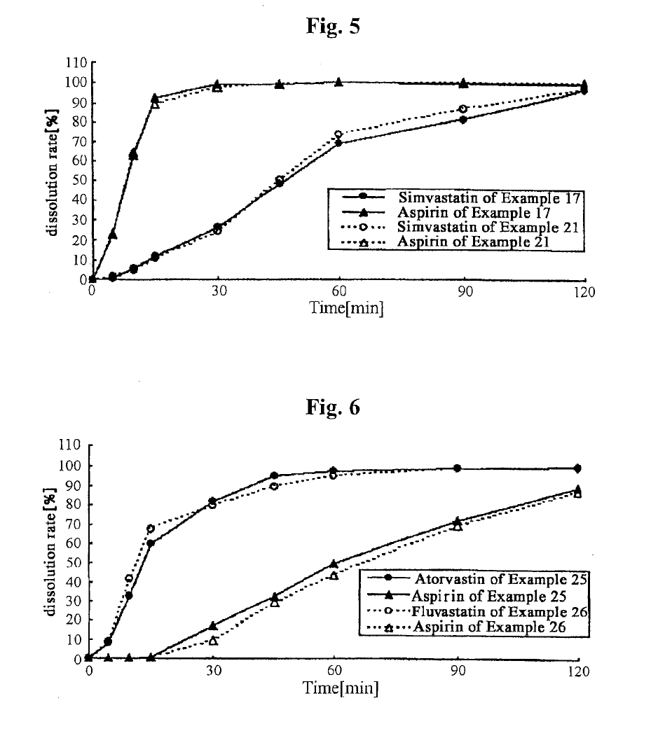

As can be seen in FIGS. 5 and 7, the combined pharmaceutical formulation of the present invention could be designed in the form of bilayer tablets, trilayer tablets or capsule formulations, and the HMG-CoA reductase inhibitor and aspirin in all the formulations could be dissolved and absorbed at different times.

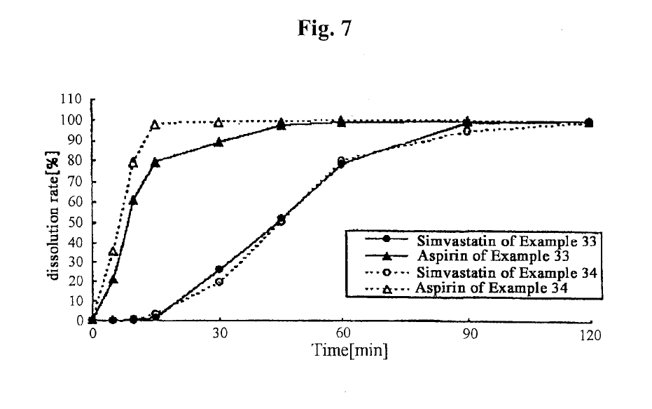

As can be seen in FIG. 6, the HMG-CoA reductase inhibitor in the combined pharmaceutical formulation of the present invention can be released at a certain time intervals from the aspirin component regardless of the kind of HMG-CoA reductase inhibitor.

### Test Example 3

### Stability Test—Accelerated & Long-Term Test

The tablets obtained in Examples above were subjected to an accelerated and a long-term storage test according to “stability test standards for drugs, etc.” described in notification No. 2006-64 of Korea Food & Drug Administration.

In a specific test method, the products prepared in Examples above were packaged in high-density polyethylene bottles and stored in a constant temperature-constant humidity chamber in conditions of temperature of 40±2° C./relative humidity of 75±5% (accelerated test conditions) and temperature of 25±2° C./relative humidity of 60±5% (long-term storage conditions). The stored tablets were taken out at a given interval of time and subjected to a quality test.

The results of the stability test are shown in Tables 8 and 9.

As can be seen in Tables 8 and 9, the tablets prepared in Examples of the present invention showed excellent stability, such that they could be stored for a long term. Particularly, the capsule formulations, press-coated tablets, bilayer tablets and trilayer tablets of the present invention, in which drugs were present in separate layers, showed excellent stability compared to that of a single preparation.

TABLE 8

| | Stability test (accelerated test conditions) | | | | | |
|---|---|---|---|---|---|---|
| | Simvastatin content (%) | | | Aspirin content (%) | | |
| | Time of | | Degra- | Time of | | Degra- |
| | test | After 3 | dation | test | After 3 | dation |
| | initiation | month | rate | initiation | month | rate |
| Example 1 | 101.0 | 98.8 | −7.9 | 103.2 | 96.1 | −8.7 |
| Example 7 | 99.9 | 98.9 | −4.8 | 101.6 | 98.7 | −4.5 |
| Example | 101.3 | 99.8 | −0.8 | 99.4 | 96.2 | −0.1 |
| 20 | | | | | | |
| Example | 101.1 | 100.2 | −4.7 | 99.3 | 96.8 | −2.5 |
| 21 | | | | | | |
| Example | 100.7 | 98.7 | −10.0 | 99.9 | 97.1 | −6.3 |
| 23 | | | | | | |
| Example | 99.1 | 97.6 | +0.1 | 99.6 | 97.4 | −0.2 |
| 33 | | | | | | |

TABLE 9

| | Stability test (long-term stability test conditions) | | | | | |
|---|---|---|---|---|---|---|
| | Simvastatin content (%) | | | Aspirin content (%) | | |
| | Time of | | Degra- | Time of | | Degra- |
| | test | After 3 | dation | test | After 3 | dation |
| | initiation | month | rate | initiation | month | rate |
| Example 1 | 101.0 | 100.6 | −7.4 | 103.2 | 101.8 | −6.7 |
| Example 7 | 99.9 | 99.8 | −3.4 | 101.6 | 100.6 | −3.0 |
| Example | 101.3 | 100.8 | −0.2 | 99.4 | 99.1 | −0.1 |
| 20 | | | | | | |
| Example | 101.1 | 100.1 | −1.7 | 99.3 | 98.7 | −1.2 |
| 21 | | | | | | |
| Example | 100.7 | 99.9 | −5.1 | 99.9 | 99.2 | −2.1 |
| 23 | | | | | | |
| Example | 99.1 | 99.7 | +0.2 | 99.6 | 99.2 | −0.1 |
| 33 | | | | | | |

### Test Example 4

### Test of Antihypertensive and Antiplatelet Aggregation Effects Using SD Rats and SHR Rats

Animal tests supporting the effects of the present invention were carried out as described in Table 10 below. The test results are shown in Tables 11 and 12.

As can be seen in Table 11 below, a group administered with simvastatin and aspirin simultaneously showed a antihypertensive effect. A vehicle group and a group administered with simvastatin alone did not show a antihypertensive effect, suggesting that the antihypertensive effect is attributable to aspirin. In addition, a group administered with drugs in the morning (administration to rats in dark conditions corresponds to administration to humans in the morning, because the biorhythm of rats is opposite to the biorhythm of humans) did not show a antihypertensive effect. Thus, it could be seen that the combined pharmaceutical formulation of the present invention had a antihypertensive effect.

As can be seen in Table 12 below, the same platelet aggregation inhibitory effect as in drug administration in the morning could be obtained in drug administration in the evening even in a reduced drug amount. Also, it was observed that the platelet aggregation inhibitory effect of aspirin increased in proportion to the dose of aspirin, but when the dose of aspirin was more than a given dose, the platelet aggregation inhibitory effect no longer increased. That is, the inventive combined pharmaceutical formulation showed a more excellent platelet aggregation inhibitory effect even with a lower dose of aspirin, and thus the superiority of the inventive antiplatelet-aggregation combined pharmaceutical formulation having the lowest dose of aspirin was proved.

TABLE 10

| Title | Tests of antiplatelet aggregation and antihypertensive effects using SD rats | | |
|---|---|---|---|
| | and SHR rats | | |
| Object | To confirm the antiplatelet aggregation through the measurement of bleeding | | |
| | time and antihypertensive effects of the inventive combined pharmaceutical | | |
| | formulation for preventing and treating cardiovascular disease through the | | |
| | measurement of bleeding time. | | |
| Test animals | rats (male), SD (9-week-old, 250-350 g) | | |
| | spontaneously hypertensive rats (SHR) rats (male, 8-week-old) | | |
| Test method | Test group (composition of | Number of | Administration |
| | Example 38) | animals | route |
| | Vehicle group (normal, SHR rats) | 10 | — |
| | Simvastatin 5 mg/kg, light | | p.o |
| | Simvastatin 5 mg/kg, aspirin 1 mg/kg, light | | |
| | Simvastatin 5 mg/kg, aspirin 5 mg/kg, light | | |
| | Simvastatin 5 mg/kg, aspirin 10 mg/kg, light | | |
| | Simvastatin 5 mg/kg, aspirin 20 mg/kg, light | | |
| | Simvastatin 5 mg/kg, aspirin 20 mg/kg, dark | | |

| | |
|---|---|
| | 1. administration route and method: oral administration was selected, and |
| | the test sample was orally administered into the stomach using a sonde for |
| | oral administration and a syringe. |
| | 2. reason for selection of administration route: the test sample was orally |
| | administered, because the clinical application route of the test sample was |
| | an oral route. |
| | 3. administration time: a group administered in the morning, and a group |
| | administered in the evening. |
| | 4. dosage |
| | 4.1 simvastatin: 5 mg/kg |
| | 4.2 aspirin: 1 mg/kg, 2.5 mg/kg, 5 mg/kg, 10 mg/kg, 20 mg/kg. |
| | 5. number of administration: administered once. |
| | 6. environmental conditions: 23 ± 2° C., relative humidity: 50 ± 10%, a |
| | 12-hr light/12-hr dark cycle. |
| Evaluation | <Measurement of blood pressure> |
| method | 1. test method |
| | 1.1 a rat was placed in each rat holder of a blood pressure monitor. |
| | 1.2 the blood pressure of the tail was measured using a cuff-pulse sensor. |
| | 1.3 one to three measurements were performed and averaged. |
| | 2. items measured: heart rate (HR), systolic blood pressure (SBP), mean |
| | blood pressure (MBP), and diastolic blood pressure.(DBP) |
| | 3. measurement time |
| | 3.1 blood pressure was measured before administration of the test sample. |
| | 3.2 blood pressure at 20 hr after administration of the test sample was |
| | measured. |
| | 3.3 blood pressure for 1, 2 and 5 days after administration of the test |
| | sample was measured. |
| | <Measurement of bleeding time> |
| | 1. At 2 hrs after drug administration, rats were anesthetized, and then a tail |
| | portion of 2 mm from the end of the tail was removed with a sterilized |
| | surgical knife. |
| | 2. blood formed at the tail end was absorbed by absorption paper in a 30- |
| | sec interval. |
| | 3. the time until bleeding was stopped was recorded. |

The results of the animal tests are shown in Tables 11 and 12.

TABLE 11
Day 0

| | Systolic blood | Mean blood | Diastolic blood |
|---|---|---|---|
| | pressure (SBP) | pressure (MBP) | pressure (DBP) |
| Normal rats | 125.0 ± 7.5 | 95.5 ± 9.7 | 80.8 ± 12.6 |
| SHR rats | 173.5 ± 11.4 | 142.6 ± 17.2 | 128.7 ± 11.9 |
| Simvastatin | 171.0 ± 8.7 | 139.0 ± 12.4 | 124.3 ± 15.5 |
| Simvastatin 5 mg/kg, | 164.6 ± 15.3 | 128.0 ± 11.7 | 110.2 ± 11.8 |
| aspirin 1 mg/kg, light | | | |
| Simvastatin 5 mg/kg, | 166.8 ± 14.0 | 130.6 ± 12.6 | 112.0 ± 12.4 |
| aspirin 5 mg/kg, light | | | |
| Simvastatin 5 mg/kg, | 166.4 ± 7.1 | 124.4 ± 7.2 | 103.4 ± 8.4 |
| aspirin 10 mg/kg, | | | |
| light | | | |
| Simvastatin 5 mg/kg, | 167.8 ± 7.9 | 137.4 ± 10.1 | 107.9 ± 18.4 |
| aspirin 20 mg/kg, | | | |
| light | | | |
| Simvastatin 5 mg/kg, | 163.6 ± 8.7 | 127.4 ± 12.9 | 109.2 ± 19.7 |
| aspirin 20 mg/kg, | | | |
| dark | | | |

Day 5

| | Systolic blood | Mean blood | Diastolic blood |
|---|---|---|---|
| Group | pressure (SBP) | pressure (MBP) | pressure (DBP) |
| Normal rats | 131.8 ± 3.8 | 94.5 ± 6.6 | 80.5 ± 13.6 |
| SHR rats | 174.7 ± 13.3 | 135.9 ± 21.4 | 127.6 ± 19.5 |
| Simvastatin | 173.9 ± 9.2 | 131.3 ± 12.6 | 125.1 ± 17.1 |
| Simvastatin 5 mg/kg, | 162.2 ± 10.4 | 122.0 ± 9.7 | 108.1 ± 12.3 |
| aspirin 1 mg/kg, light | | | |
| Simvastatin 5 mg/kg, | 163.4 ± 11.9 | 120.2 ± 23.4 | 108.7 ± 11.8 |
| aspirin 5 mg/kg, light | | | |
| Simvastatin 5 mg/kg, | 158.1 ± 7.8 | 123.0 ± 18.6 | 100.1 ± 9.1 |
| aspirin 10 mg/kg, | | | |
| light | | | |
| Simvastatin 5 mg/kg, | 156.4 ± 12.9 | 121.1 ± 17.4 | 99.7 ± 11.8 |
| aspirin 20 mg/kg, | | | |
| light | | | |
| Simvastatin 5 mg/kg, | 165.4 ± 12.3 | 132.3 ± 14.6 | 110.1 ± 16.5 |
| aspirin 20 mg/kg, | | | |
| dark | | | |

TABLE 12

| | Bleeding time |
|---|---|
| Normal rats | 750 ± 468 sec |
| Simvastatin, light conditions | 782 ± 429 sec |
| Simvastatin 5 mg/kg, aspirin 1 mg/kg, light conditions | 996 ± 314 sec |
| Simvastatin 5 mg/kg, aspirin 2.5 mg/kg, light conditions | 1218 ± 459 sec |
| Simvastatin 5 mg/kg, aspirin 5 mg/kg, light conditions | 1369 ± 618 sec |
| Simvastatin 5 mg/kg, aspirin 10 mg/kg, light conditions | 1419 ± 759 sec |
| Simvastatin 5 mg/kg, aspirin 20 mg/kg, light conditions | 1331 ± 719 sec |
| Simvastatin 5 mg/kg, aspirin 20 mg/kg, dark conditions | 971 ± 219 sec |

### Test Example 5

### Test of Cholesterol Concentration Lowering

In this Test Example, animal tests supporting the effects of the present invention were carried out as described in Table 13 below. The test results are shown in Table 14 below.

As can be seen in Table 14, the blood lipid lowering effect of the inventive combined pharmaceutical formulation, comprising a HMG-CoA reductase inhibitor and aspirin, was equal to that of a single preparation. In addition, the vehicle group, in which the drugs were simultaneously released in vivo, showed a decrease in the blood lipid concentration lowering effect, but the inventive combined pharmaceutical formulation did not show this decrease, suggesting that, in the inventive combined pharmaceutical formulation, the decrease in therapeutic effect by the interaction between the two drug components did not occur.

TABLE 13

| | |
|---|---|
| Title | Test of antihyperlipidemia effects of simvastatin/aspirin |
| | combined pharmaceutical formulation and simvastatin single |
| | preparation in hyperlipidemia rats |
| Object | To confirm the hyperlipidemia therapeutic effect of a |
| | simvastatin/aspirin combined pharmaceutical formulation by |
| | comparison with the therapeutic effect of a simvastatin |
| | single preparation. |
| Test | Rats having induced hyperlipidemia |
| animal | |
| Test | 1. administration route and method: oral administration was |
| method | selected, and the test sample was orally administered into the |
| | stomach using a sonde for oral administration and a syringe. |
| | 2. reason for selection of administration route: the test sample |
| | was orally administered, because the clinical application |
| | route of the test sample was an oral route. |
| | 3. dosage |
| | 3.1 simvastatin: 5 mg/kg |
| | 3.2 aspirin: 5 mg/kg |
| | 4. preparations administered |
| | 4.1 vehicle: simvastatin + aspirin solution |
| | 4.2 group administered with simvastatin: simvastatin |
| | 4.3 test group: the capsule formulation of Example 37 |
| | 5. number of administration: administered once a day for 7 |
| | days |
| | 6. environmental conditions: 23 ± 2° C., relative humidity: |
| | 50 ± 10%, a 12-hr light/12-hr dark cycle. |
| Evaluation | LDL-C was measured at 4 hrs after final administration |
| method | |

TABLE 14

| | Measurement of LDL concentration | |
|---|---|---|
| Group | Before administration | After administration |
| Vehicle | 170.16 | 113.6 |
| Group administered | 167 ± 21.1 | 88 ± 25.7 |
| with simvastatin | | |
| Test group | 175 ± 33.7 | 94 ± 19.6 |

### Example 6

### Test of Comparison of Gastric Mucosal Injury in Rats

Animal tests supporting the effects of the present invention were carried out as described in Table 15. The test results are shown in Table 16 below.

As can be seen in Table 16, the probability of occurrence of gastrointestinal disturbance increased in proportion to an increase in the dose of aspirin. This suggests that the inventive combined pharmaceutical formulation comprising a HMG-CoA reductase inhibitor and aspirin shows significantly reduced gastrointestinal disturbance through the decrease in the dose of aspirin and administration at optimal time.

TABLE 15

| Title | Evaluation of gastrointestinal disorders through observation of gastric | | | |
|---|---|---|---|---|
| | mucosal bleeding upon administration of simvastatin/aspirin combined | | | |
| | pharmaceutical formulation | | | |
| Object | To examine whether the combined pharmaceutical formulation of the | | | |
| | present invention causes gastrointestinal disturbance | | | |
| Test animals | rats (male, 9-week-old, 250-350 g) | | | |
| Test method | Test group (composition of | | Animals | Administration |
| | Example 8) | Example | (number) | route |
| | Normal rat (administered with saline) | — | 10 | p.o |
| | SHR rats (administered with saline) | | | |
| | Simvastatin 5 mg/kg, aspirin | 8 | | |
| | 1 mg/kg, light | | | |
| | Simvastatin 5 mg/kg, aspirin | | | |
| | 5 mg/kg, light | | | |
| | Simvastatin 5 mg/kg, aspirin | | | |
| | 10 mg/kg, light | | | |
| | Simvastatin 5 mg/kg, aspirin | | | |
| | 20 mg/kg, light | | | |
| | Simvastatin 5 mg/kg, aspirin | | | |
| | 40 mg/kg, light | | | |
| | Simvastatin 5 mg/kg, aspirin | | | |
| | 80 mg/kg, light | | | |
| | 1. administration route and method: oral administration was selected, and | | | |
| | the test sample was orally administered into the stomach using a sonde for | | | |
| | oral administration and a syringe. | | | |
| | 2. reason for selection of administration route: the test sample was orally | | | |
| | administered, because the clinical application route of the test sample was | | | |
| | an oral route. | | | |
| | 3. dosage | | | |
| | 3.1 simvastatin: 5 mg/kg | | | |
| | 3.2 aspirin: 1 mg/kg, 5 mg/kg, 10 mg/kg, 20 mg/kg, 40 mg/kg, 80 mg/kg. | | | |
| | 5. number of administration: administered once a day for 10 days. | | | |
| | 6. environmental conditions: 23 ± 2° C., relative humidity: 50 ± 10%, a | | | |
| | 12-hr light/12-hr dark cycle. | | | |
| Evaluation | 1. At 3 hrs after final administration of the drug, the stomach of the rats | | | |
| method | was incised. | | | |
| | 2. The stomach was observed to measure the number and range of | | | |
| | bleeding sites. | | | |
| | 3. Based on the measurement results, the index of gastrointestinal | | | |
| | disturbance was calculated. | | | |
| | *index of gastrointestinal disturbance = sum of indexes of gastric mucosal | | | |
| | bleeding/number of animals | | | |
| | *index of gastric mucosal bleeding | | | |
| | Index of gastric | | | |
| | mucosal bleeding | | | |

| | |
|---|---|
| 0 | No injury |
| 1 | Less than 5 injury sites |
| 2 | More than 5 injury sites |
| 3 | The size of injuries connected is less |
| | than 2 mm or the size of a unit injury is |
| | more than 2 mm. |
| 4 | The number of injuries connected is |
| | very large. |

TABLE 16

| | Index of gastric |
|---|---|
| | mucosal bleeding |
| Normal rats (administered with saline) | 0.7 |
| SHR rats (administered with saline) | 0.9 |
| Simvastatin 5 mg/kg, aspirin 1 mg/kg, light | 1.4 |
| Simvastatin 5 mg/kg, aspirin 5 mg/kg, light | 1.8 |
| Simvastatin 5 mg/kg, aspirin 10 mg/kg, light | 1.9 |
| Simvastatin 5 mg/kg, aspirin 20 mg/kg, light | 2.7 |
| Simvastatin 5 mg/kg, aspirin 40 mg/kg, light | 3.1 |
| Simvastatin 5 mg/kg, aspirin 80 mg/kg, light | 3.2 |

1. A chronotherapeutically combined pharmaceutical formulation for preventing or treating cardiovascular disease, comprising, as active ingredients, a HMG-CoA reductase inhibitor component selected from the group consisting of a HMG-CoA reductase inhibitor, pharmaceutically acceptable salts thereof and isomers thereof and an aspirin component selected from the group consisting of aspirin and pharmaceutically acceptable salts thereof, wherein the HMG-CoA reductase inhibitor component and the aspirin components are released at different absorption locations or are sequentially released at the same absorption location.

2. The chronotherapeutically combined pharmaceutical formulation according to claim 1, wherein the aspirin component is released within 15 minutes to 4 hours after the HMG-CoA reductase inhibitor component is released.

3. The combined pharmaceutical formulation according to claim 1, wherein, as measured in a dissolution test conducted in simulated intestinal fluid containing 1% sodium lauryl sulfate, at 30 minutes after the start of dissolution, not less than 75% of the HMG-CoA reductase inhibitor component is dissolved, and less than 40% of the aspirin component is dissolved.

4. The combined pharmaceutical formulation according to claim 1, wherein the HMG-CoA reductase inhibitor component is released within 15 minutes to 4 hours after the aspirin component is released.

5. The combined pharmaceutical formulation according to claim 1, wherein, as measured in a dissolution test conducted in simulated intestinal fluid containing 1% sodium lauryl sulfate, at 15 minutes after the start of dissolution, not less than 70% of the aspirin component is dissolved, and less than 40% of the HMG-CoA reductase inhibitor component is dissolved.

6. The combined pharmaceutical formulation according to claim 1, wherein the absorption location of the active ingredients is selected from oral mucosa, stomach, small intestines and large intestines.

7. The combined pharmaceutical formulation according to claim 1, wherein the HMG-CoA reductase inhibitor is selected from the group consisting of simvastatin, atorvastatin, pravastatin, fluvastatin, rosuvastatin, cerivastatin, pharmaceutically acceptable salts thereof and isomers thereof.

8. The combined pharmaceutical formulation according to claim 7, wherein the HMG-CoA reductase inhibitor is simvastatin or atorvastatin.

9. The combined pharmaceutical formulation according to claim 1, wherein the content of the HMG-CoA reductase inhibitor component is 0.5-80.0 mg, and the content of the aspirin component is 20.0-700.0 mg.

10. The combined pharmaceutical formulation according to claim 9, wherein the content of the aspirin component is not less than 20.0 mg and less than 75.0 mg.

11. The combined pharmaceutical formulation of claim 10, wherein the content of the aspirin component is 20.0-40.0 mg.

12. The combined pharmaceutical formulation according to claim 1, further comprising, as a release-controlling substance of the active ingredients, at least one selected from a water-soluble polymer, a water˜insoluble polymer, an enteric polymer, an oil substance and gum.

13. The combined pharmaceutical formulation according to claim 12, wherein the release-controlling substance is contained in an amount of 0.1-100 parts by weight based on 1 part by weight of the active ingredient which is released slower.

14. The combined pharmaceutical formulation according to claim 12, wherein the water-soluble polymer is at least one selected from the group consisting of: a water-soluble cellulose ether selected from methylcellulose, hydroxypropylcellulose and hydroxypropylmethylcellulose; a water-soluble polyvinyl derivative selected from polyvinyl pyrrolidone and polyvinyl alcohol; and an alkylene oxide polymer selected from polyethylene glycol and polypropylene glycol.

15. The combined pharmaceutical formulation according to claim 12, wherein the water-insoluble polymer is at least one selected from the group consisting of a water-insoluble cellulose ether selected from ethyl cellulose and cellulose acetate; and a water-insoluble acrylic copolymer selected from an ethylacrylate/methylmethacrylate/trimethylammonium chloride ethyl methacrylate copolymer and a methylmethacrylate/ethylacrylate/trimethylammonium chloride ethyl copolymer.

16. The combined pharmaceutical formulation according to claim 12, wherein the enteric polymer is at least one selected from the group consisting of: an enteric cellulose derivative selected from hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose benzoate phthalate, cellulose propionate phthalate, methylcellulose phthalate, carboxymethylethylcellulose and ethylhydroxyethylcellulose phthalate; an enteric acrylic acid copolymer selected from a styrene/acrylic acid copolymer, a methylacrylate/acrylic acid copolymer, a methylacrylate/methacrylic acid copolymer, a butylacrylate/styrene/acrylic acid copolymer, a methacrylic acid/ethylmethacrylate copolymer, a methacrylic acid/ethylacrylate copolymer and a methylacrylate/methacrylic acid/octylacrylate copolymer; an enteric maleic acid copolymer selected from a vinylacetate/maleic anhydride copolymer, a styrene/maleic anhydride copolymer, a styrene/maleic monoester copolymer, a vinylmethylether/maleic anhydride copolymer, an ethylene/maleic anhydride copolymer, a vinylbutylether/maleic anhydride copolymer, an acrylonitrile/methylacrylate/maleic anhydride copolymer and a butyl acrylate/styrene/maleic anhydride copolymer; and an enteric polyvinyl derivative selected from polyvinylalcohol phthalate, polyvinylacetal phthalate, polyvinylbutyrate phthalate and polyvinylacetacetal phthalate.

17. The combined pharmaceutical formulation according to claim 12, wherein the oil substance is at least one selected from the group consisting of: a fatty acid or fatty acid ester selected from glyceryl palmitostearate, glyceryl stearate, glyceryl behenate, cetyl palmitate, glyceryl monooleate and stearic acid; a fatty acid alcohol selected from cetostearyl alcohol, cetyl alcohol and stearyl alcohol; and a wax selected from carnauba wax, beeswax and microcrystalline wax.

18. The combined pharmaceutical formulation according to claim 12, wherein the gum is at least one selected from the group consisting of guar gum, locust bean gum, tragacantha, carrageenan, acacia gum, arabia gum, gellan gum, xanthan gum and pectin.

19. The combined pharmaceutical formulation according to claim 1, which is in the form of a uncoated tablet, a film-coated tablet, a multilayer tablet, a press-coated tablet, a capsule formulation or a kit.

20. The combined pharmaceutical formulation according to claim 19, wherein the multilayer tablet is a trilayer tablet consisting of a first layer of the HMG-CoA reductase inhibitor component, a second layer of placebo, and a third layer of the aspirin component.

21. The combined pharmaceutical formulation according to claim 19, wherein the capsule formulation comprises at least one selected from the group consisting of granules, tablets, mini-tablets and pellets, which comprise the HMG-CoA reductase inhibitor component and the aspirin component, in the capsule.

22. The combined pharmaceutical formulation according to claim 19, wherein the kit is packaged such that the HMG-CoA reductase inhibitor component and the aspirin component can be simultaneously administered.

23. The combined pharmaceutical formulation of claim 19, wherein the film-coated tablet comprises a coating layer, which includes a film-forming agent, a filmforming aid or mixture thereof.

24. The combined pharmaceutical formulation of claim 10, which is administered in the evening.

25. The combined pharmaceutical formulation of claim 1, wherein the HMG CoA reductase inhibitor is simvastatin or atorvastatin and the formulation is in the form of a capsule formulation or a trilayer tablet.
